(19) United States
(12) Patent Application Publication
(10) Pub. No.: US 2023/0140823 A1
(43) Pub. Date: May 4, 2023

(54) A stabilized protein of interest.

(71) Applicant: Micreos Human Health B.V., Den Haag (NL)

(72) Inventor: Fritz Eichenseher, Den Haag (NL)

(73) Assignee: Micreos Human Health B.V., Den Haag (NL)

(21) Appl. No.: 17/912,050

(22) PCT Filed: Mar. 18, 2021
(86) PCT No.: PCT/EP2021/056910

§ 371 Date: Sep. 16, 2022

(30) Foreign Application Priority Data
Mar. 19, 2020 (EP) 20164184.2

(51) Int. Cl. C12N 9/96; A61K 38/46; A61K 47/46; A61K 9/00; A61K 9/06; A61K 9/14; A61K 9/19; A61P 17/00; C12N 9/36

(52) U.S. Cl. CPC C12N 9/96; A61K 38/465; A61K 47/46; A61K 9/0014; A61K 9/06; A61K 9/148; A61K 9/19; A61P 17/00; C12N 9/2462; C12Y 302/01017

(57) ABSTRACT

The present invention relates to the field of medicine, specifically to the field of treatment of a malignant condition associated with infection with a bacterium that aggravates and/or induces proliferation of the malignant conditions.

### FIELD OF THE INVENTION

The present invention relates to the field of molecular biology, specifically the field of enzymes.

### BACKGROUND OF THE INVENTION

Dermatitis, also known as eczema, is a group of diseases that result in inflammation of the skin (Nedorost et al, 2012). These diseases are characterized by itchiness, red skin and a rash. In cases of short duration, there may be small blisters, while in long-term cases the skin may become thickened. The area of skin involved can vary from small to the entire body (Handout on Health: Atopic Dermatitis (A type of eczema)”. NIAMS. May 2013). Dermatitis is a group of skin conditions that includes atopic dermatitis, allergic contact dermatitis, irritant contact dermatitis and stasis dermatitis. The exact cause of dermatitis is often unclear. Cases may involve a combination of irritation, allergy and poor venous return. The type of dermatitis is generally determined by the person's history and the location of the rash. For example, irritant dermatitis often occurs on the hands of people who frequently get them wet. Allergic contact dermatitis occurs upon exposure to an allergen, causing a hypersensitivity reaction in the skin. State of art treatment of atopic dermatitis is typically with moisturizers and steroid creams (McAleer et al, 2012). The steroid creams are generally of mid- to high strength and are preferably used for less than two weeks at a time as side effects can occur (Habif et al, 2015). Antibiotics are typically used if there are signs of skin infection. Contact dermatitis is typically treated by avoiding the allergen or irritant (Mowad et al, 2016; Laruti et al, 2015). Anti-histamines may help with sleep and to decrease nighttime scratching. Dermatitis symptoms may vary with different forms of the condition. They range from skin rashes to bumpy rashes or including blisters. Although every type of dermatitis may have different symptoms, there are certain signs that are common for all of them, including redness of the skin, swelling, itching and skin lesions with sometimes oozing and scarring. Also, the area of the skin on which the symptoms appear tends to be different with every type of dermatitis, whether on the neck, wrist, forearm, thigh or ankle. Although the location may vary, the primary symptom of this condition is itchy skin.

Although the symptoms of atopic dermatitis vary from person to person, the most common symptoms are dry, itchy, red skin. Typical affected skin areas include the folds of the arms, the back of the knees, wrists, face and hands. Dermatitis was estimated to affect 245 million people globally in 2015 (Lancet. 388 (10053): 1545-1602). Atopic dermatitis is the most common type and generally starts in childhood. In the United States, it affects about 10-30% of people.

Recently, a novel combination treatment of dermatitis using an anti-inflammatory first compound in combination with a second compound specifically targeting a bacterial cell, said second compound preferably being an (chimeric) bacteriophage endolysin specifically targeting Staphylococcus aureus (WO2015005787, which is herein incorporated by reference).

Oats have also been used for the treatment of dermatitis, at least to alleviate the symptoms. Oats (Avena sativa) have been cultivated since the Bronze Age, and have been used in traditional medicine for centuries. As a topical treatment, colloidal oatmeal has emollient and anti-inflammatory properties, and is commonly used for skin rashes, erythema, burns, itch, and eczema.

There is no cure for eczema. Prolonged use of topical corticosteroids is thought to increase the risk of side effects, the most common of which is the skin becoming thin and fragile (atrophy). Because of this, if used on the face or other delicate skin, a low-strength steroid should be used or applied less frequently. Additionally, high-strength steroids used over large areas, or under occlusion, may be absorbed into the body, causing hypothalamic-pituitary-adrenal axis suppression (HPA axis suppression). The effectiveness of antibiotic treatments varies from person to person. The well-known disadvantages of conventional antibiotics are a-specificity, i.e. also non-pathogenic and/or beneficial bacteria are killed, and the risk of developing resistance, not only by the target bacteria but possibly also by other pathogenic bacteria. Furthermore, conventional, systemic antibiotic treatment can interact with other drugs, including contraceptive pills.

Altogether, there is a need for improved treatment of eczema.

### DESCRIPTION OF THE INVENTION

Endolysins lose their activity over time when in aqueous solutions. When bringing the protein in a lyophilized form, the inventors found that using oatmeal as a carrier, the stability of the endolysin surprisingly increased. The inventors additionally established that other proteins can be stabilized as well.

Accordingly, in a first aspect there is provided for a method for stabilizing a protein of interest, comprising contacting the protein with a cereal meal or variant thereof. The method is herein referred to for all embodiments as a method as disclosed herein or as the method.

Further provided is a non-aqueous composition comprising a protein of interest and a cereal meal or variant thereof. The composition is herein referred to for all embodiments as a composition as disclosed herein or as the composition.

Non-aqueous is herein construed as that the composition contains substantially no water; preferably the amount of water is at most 10% (as weight percent), 9%, 8%, 7%, 6%, 5%, 4%, 3%, 2%, 1%, 0.5%, 0.4%, 0.3%, 0.2%, or at most 0.1%.

In the method or composition, the protein of interest may be any protein, such as a peptide, oligopeptide, a polypeptide or a mature protein. The protein may be a bacteriocin or an antifungal protein, preferably a bacteriocin as defined in the section “Definitions” herein. Preferably, the protein is an enzyme. The enzyme may be any enzyme. The enzyme may be an antibacterial enzyme, such as an endolysin, such as a bacteriophage endolysin or a recombinant bacteriophage endolysin. An antibacterial enzyme may be one selected from the group of lysozyme, phospholipase A₂ and gastric enzymes.

In the method or composition, the bacteriophage endolysin or recombinant endolysin may be any bacteriophage endolysin known to the persons skilled in the art. Herein, the terms bacteriophage lysin, bacteriophage endolysin and endolysin are used interchangeably. An endolysin may be selected from the group of endolysins defined in WO2011/023702, WO2012/146738, WO2003/082184 (BIOSYNEX), WO2010/011960 (Donovan), WO2010/149795, WO2010/149792, WO2012/094004, WO2011/023702, WO2011/065854, WO2011/076432, WO2011/134998, WO2012/059545, WO2012/085259, WO2012146738, WO2018/091707, Exebacase™ (Lysin CF-301; Antimicrobial Agents and Chemotherapy, 2019, vol 63:6, 1-17), SAL200™ (Antimicrobial Agents and Chemotherapy, 2018, vol 62:10, 1-10), Auresine™ (Sigma-Aldrich SAE0083), and Ectolysin™ P128 (Antimicrobial Agents and Chemotherapy, 2018, vol 62:2, 1-10), which are herein incorporated by reference in their entirety.

In the method or composition, the endolysin may be a Staphylococcus-specific endolysin, meaning that it will lyse Staphylococcus, such as Staphylococcus aureus, efficiently but does not substantially lyse other bacteria than Staphylococcus or Staphylococcus aureus. In an embodiment, the endolysin will lyse Staphylococcus aureus, but not Staphylococcus epidermidis. Most native Staphylococcus bacteriophage endolysins exhibiting peptidoglycan hydrolase activity consist of a C-terminal cell wall-binding domain (CBD), a central N-acetylmuramoyl-L-Alanine amidase domain, and an N-terminal Alanyl-glycyl endopeptidase domain with cysteine, histidine-dependent amidohydrolases/peptidase (CHAP) homology, or in case of Ply2638, of an N-terminal glycyl-glycine endopeptidase domain with Peptidase_M23 homology, the latter three domains exhibiting peptidoglycan hydrolase activity each with distinct target bond specificity and generally named as enzymatically active domains. The Ply2638 endolysin is set forward in SEQ ID NO: 1 and SEQ ID NO: 2 (see Table 1); several endolysin domains are set forward in SEQ ID NO: 3 to SEQ ID NO: 18 (see Table 1), these domains are preferred domains. The endolysin may be a recombinant endolysin, such as a recombinant Staphylococcus-specific endolysin, in particular a recombinant Staphylococcus-specific chimeric endolysin comprising one or more heterologous domains. In general, endolysins are comprised of different subunits (domains); e.g. a cell wall-binding domain (CBD) and one or more enzymatic domains having peptidoglycan activity, such as an amidase domain, an M23 peptidase domain and a CHAP (cysteine, histidine-dependent amidohydrolases/peptidases) domain. An example of a Staphylococcus-specific chimeric endolysin comprising one or more heterologous domains is an endolysin comprising an Amidase domain of bacteriophage Ply2638, an M23 peptidase domain of lysostaphin (S. simulans) and a cell wall-binding domain of bacteriophage Ply2638. Such Staphylococcus-specific chimeric endolysin is a preferred endolysin and is extensively described in WO2012/150858, which is herein incorporated by reference in its entirety. Other preferred endolysins are extensively described in WO2013/169104, which is herein incorporated by reference in its entirety. Other preferred endolysins according to the invention are extensively described in WO2016/142445, which is herein incorporated by reference in its entirety. Other preferred endolysins according to the invention are extensively described in WO2017/046021, which is herein incorporated by reference in its entirety. The endolysin may further be one selected from the group consisting of the endolysins depicted as SEQ ID NO: 19 to SEQ ID NO: 75 in Table 1. It should be noted that endolysins such as depicted in Table 1 can be used with or without tag (HXa).

In the method or composition, the endolysin may comprise a domain having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity with a domain depicted in WO2012/150858, WO2013/169104, WO2016/142445, WO2017/046021 or with a domain in an endolysin depicted in any of SEQ ID NO: 3 to SEQ ID NO: 18 (see Table 1).

In the method or composition, the endolysin may have at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity with an endolysin depicted in WO2012/150858, WO2013/169104, WO2016/142445, WO2017/046021 or with an endolysin depicted in any of SEQ ID NO: 1, 2 and SEQ ID NO: 19 to SEQ ID NO: 75 (see Table 1). It should be noted that endolysins such as depicted in Table 1 can be used with or without tag (HXa).

The person skilled in the art will comprehend that mixes of different endolysins may be used in the, e.g. a mix comprising two, three or four endolysins specified herein.

A cereal is any grass cultivated (grown) for the edible components of its grain (botanically, a type of fruit called a caryopsis), composed of the endosperm, germ, and bran. The term may also refer to the resulting grain itself (specifically “cereal grain”). Cereal grain crops are grown in greater quantities and provide more food energy worldwide than any other type of crop and are therefore staple crops. Edible grains from other plant families, such as buckwheat (Polygonaceae), quinoa (Amaranthaceae) and chia (Lamiaceae), are referred to as pseudocereals.

In their natural, unprocessed, whole grain form, cereals are a rich source of vitamins, minerals, carbohydrates, fats, oils, and protein. When processed by the removal of the bran, and germ, the remaining endosperm is mostly carbohydrate. In some developing countries, grain in the form of rice, wheat, millet, or maize constitutes a majority of daily sustenance.

Colloidal oatmeal is the finely ground whole oat kernel or groat, and is an active natural ingredient covered by the FDA OTC Skin Protectant monograph in the US (The United States Pharmacopeial Convention, Interim Revision Announcement; Official Jan. 1, 2013). Typically, the oat grain is ground and processed until no more than 3% of the total particles exceed 150 μm and no more than 20% exceeds 75 μm. The composition of colloidal oatmeal largely consists of starch (65-85%), protein (15-20%), lipids (3-11%), fiber (5%) and β-glucans (5%). Oat lipids are primarily composed of triglycerides, along with polar lipids and unsaturated free fatty acids. Oat triglycerides are rich in omega-3 linoleic and omega-6 linolenic acids and essential fatty acids which are necessary for normal mammalian health and important for skin barrier function. In addition, oat lipids contain important mammalian cell membrane components, such as phospholipids, glycolipids, and sterols. Lipid oxidation protection is supplied by mixed tocopherols (vitamin E) and tocotrienols. Colloidal oatmeal is also a rich source of phenolic antioxidants and saponins. Avenanthramides, nitrogen-containing phenolic compounds specific to oats, are potent antioxidants and anti-inflammatory agents that have been previously shown to inhibit NF-κB and IL-8 release in a dose dependent manner. Saponins are glycosylated metabolites which help to protect oat plants from disease and which can also help create stable emulsions when colloidal oatmeal is used in a formulation.

In the method or composition, the cereal meal or variant thereof, may comprise in weight between about 50% to about 85% carbohydrates, between about 10 and about 25% protein, between about 0% and 12% lipids, between about 0% and 10% beta-glucans and between about 0% and about 15% fibre. The cereal meal of variant thereof may comprise in weight about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or about 85% carbohydrates. The cereal meal of variant thereof may comprise in weight about 10, 11, 12, 13, 14, 15, 16, 17, 18, 19, 20, 21, 22, 23, 24 or about 25% protein. The cereal meal of variant thereof may comprise in weight about 0, 1, 2, 3, 4, 5, 6, 7, 8, 9, 10, 11, or about 12% lipids. The cereal meal of variant thereof may comprise in weight about 0, 1, 2, 3, 4, 5, 6, 7, 8, 9, or about 10% beta-glucans. The cereal meal of variant thereof may comprise in weight about 0, 1, 2, 3, 4, 5, 6, 7, 8, 9, 10, 11, 12, 13, 14, or about 15% fibre.

In the method or composition, the cereal meal of variant thereof may comprise in weight about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or about 85% carbohydrates. The cereal meal of variant thereof may comprise in weight 10, 11, 12, 13, 14, 15, 16, 17, 18, 19, 20, 21, 22, 23, 24 or 25% protein. The cereal meal of variant thereof may comprise in weight 0, 1, 2, 3, 4, 5, 6, 7, 8, 9, 10, 11, or 12% lipids. The cereal meal of variant thereof may comprise in weight 0, 1, 2, 3, 4, 5, 6, 7, 8, 9, or 10% beta-glucans. The cereal meal of variant thereof may comprise in weight 0, 1, 2, 3, 4, 5, 6, 7, 8, 9, 10, 11, 12, 13, 14, or 15% fibre. The cereal meal may comprise in weight about 65, 66, 67, 68, 69, 70, 71, 72, 73, 74, 75, 76, 77, 78, 79, 80, 81, 82, 83, 84, or about 85% carbohydrates, about 15, 16, 17, 18, 19, or about 20% protein, about 3, 4, 5, 6, 7, 8, 9, 10, or about 11% lipids, about 5% beta-glucans and about 11% fibre. The cereal meal may comprise in weight 65, 66, 67, 68, 69, 70, 71, 72, 73, 74, 75, 76, 77, 78, 79, 80, 81, 82, 83, 84, or 85% carbohydrates, 15, 16, 17, 18, 19, or about 20% protein, 3, 4, 5, 6, 7, 8, 9, 10, or 11% lipids, 5% beta-glucans and 11% fibre. The cereal meal may comprise in weight about 66% carbohydrates, about 17% protein, about 7% lipids, about 5% beta-glucans and about 11% fibre. The cereal meal may comprise in weight 66% carbohydrates, 17% protein, 7% lipids, 5% beta-glucans and 11% fibre.

In the method or composition, the cereal meal or variant thereof may be prepared from a cereal selected from the group consisting of maize, rice, wheat, barley, sorghum, millet, oats, rye, triticale, quinoa, spelt and fonio.

In the method or composition, the cereal meal may be oat meal, such as colloidal oat meal, preferably a commercially available colloidal oat meal such as: Oat Com™, Oat Silk™, or DermiVeil™, see Table 4 for further information. Colloidal oatmeal is the finely ground whole oat kernel or groat, and is an active natural ingredient covered by the FDA OTC Skin Protectant monograph in the US. Typically, the oat grain is ground and processed until no more than 3% of the total particles exceed 150 μm and no more than 20% exceeds 75 μm. The composition of colloidal oatmeal largely consists of starch (65-85%), protein (15-20%), lipids (3-11%), fiber (5%) and β-glucans (5%). Oat lipids are primarily composed of triglycerides, along with polar lipids and unsaturated free fatty acids. Oat triglycerides are rich in omega-3 linoleic and omega-6 linolenic acids and essential fatty acids which are necessary for normal mammalian health and important for skin barrier function. In addition, oat lipids contain important mammalian cell membrane components, such as phospholipids, glycolipids, and sterols. Lipid oxidation protection is supplied by mixed tocopherols (vitamin E) and tocotrienols. Colloidal oatmeal is also a rich source of phenolic antioxidants and saponins. Avenanthramides, nitrogen-containing phenolic compounds specific to oats, are potent antioxidants and anti-inflammatory agents that have been previously shown to inhibit NF-κB and IL-8 release in a dose dependent manner. Saponins are glycosylated metabolites which help to protect oat plants from disease and which can also help create stable emulsions when colloidal oatmeal is used in a formulation.

In an embodiment, in the method or the composition, the protein of interest and the cereal meal or variant thereof are mixed in an aqueous liquid, which is subsequently lyophilized. The person skilled in the art knows how to lyophilize a compound and will use a state of the art method to lyophilize the mixture.

In a second aspect, there is provided, a stabilized protein obtainable or obtained by a method according to the first aspect. The stabilized protein is herein referred for all embodiments as the protein. The features of all embodiments of the second aspect are preferably the features of the embodiments of the first aspect. Also provided is the stabilized protein comprised in a non-aqueous composition. The composition may be in any form known to the person skilled in the art, such as a cream, ointment, balm, unguent, or salve, typically a cream.

Further provided is the use of a cereal meal or variant thereof as defined herein for stabilizing a protein of interest as defined herein by contacting the protein of interest with the cereal meal or variant thereof.

Further provided is a composition comprising:
cereal meal as defined herein, preferably oat meal, more preferably colloidal oat meal, even more preferably Oat Com™, Oat Silk™, or DermiVeil™, and
an antibacterial polypeptide comprising enzymatic activity as defined herein.

In a third aspect, there is provided, a method of treatment of atopic dermatitis comprising administration of a non-aqueous composition according to the first or second aspect herein to a subject in need thereof. In all embodiments herein, the subject is a vertebrate, preferably a mammal, more preferably a human. The person skilled in the art will comprehend that treatment with the non-aqueous composition may conveniently be combined with other compounds know in the art to treat atopic dermatitis.

The medical treatment as set forward here above includes a non-aqueous composition as defined herein for the manufacture of a medicament for the prevention, delay or treatment of atopic dermatitis in a subject in need thereof as well as a method for the prevention, delay or treatment of atopic dermatitis in a subject in need thereof, comprising administration of the non-aqueous composition to the subject. Administration may be in any form known to the person skilled in the art, typically the composition will be applied to the skin.

TABLE 1
Overview of sequences

| SEQ ID NO | Name construct | organism |
|---|---|---|
| 1 | Ply2638 endolysin CDS | Bacteriophage 2638A |
| 2 | Ply2638 endolysin PRT | Bacteriophage 2638A |
| 3 | CWT-LST CDS | S. simulans |
| 4 | CWT-LST PRT | S. simulans |
| 5 | CBD2638 CDS | Bacteriophage 2638A |
| 6 | CBD2638 PRT | Bacteriophage 2638A |
| 7 | CWT-NM3 CDS | S. aureus phage phiNM3 |
| 8 | CWT-NM3 PRT | S. aureus phage phiNM3 |
| 9 | CHAPK CDS | S. phage K |
| 10 | CHAPK PRT | S. phage K |
| 11 | CHAP-ϕTwort CDS | S. phage Twort |
| 12 | CHAP-ϕTwort PRT | S. phage Twort |
| 13 | M23-2638 CDS | Bacteriophage 2638A |
| 14 | M23-2638 PRT | Bacteriophage 2638A |
| 15 | M23-LST CDS | S. simulans |
| 16 | M23-LST PRT | S. simulans |
| 17 | Ami2638 CDS | Bacteriophage 2638A |
| 18 | Ami2638 PRT | Bacteriophage 2638A |
| 19 | CHAPK_CHAPK_CWT-LST CDS | artificial construct |
| 20 | CHAPK_CHAPK_CWT-LST PRT | artificial construct |
| 21 | M23-LST_M23-LST_CWT-LST CDS | artificial construct |
| 22 | M23-LST_M23-LST_CWT-LST PRT | artificial construct |
| 23 | Ami2638_ami2638_CWT-LST CDS | artificial construct |
| 24 | Ami2638_ami2638_CWT-LST PRT | artificial construct |
| 25 | HXaAmi2638_CBD2638 CDS | artificial construct |
| 26 | HXaAmi2638_CBD2638 PRT | artificial construct |
| 27 | HXaAmi2638_CWT-LST CDS | artificial construct |
| 28 | HXaAmi2638_CWT-LST PRT | artificial construct |
| 29 | HXaAmi2638_CWT-NM3 CDS | artificial construct |
| 30 | HXaAmi2638_CWT-NM3 PRT | artificial construct |
| 31 | HXaCHAPK_CBD2638 CDS | artificial construct |
| 32 | HXaCHAPK_CBD2638 PRT | artificial construct |
| 33 | HXaCHAPK_CWT-LST CDS | artificial construct |
| 34 | HXaCHAPK_CWT-LST PRT | artificial construct |
| 35 | HXaCHAPK_CWT-NM3 CDS | artificial construct |
| 36 | HXaCHAPK_CWT-NM3 PRT | artificial construct |
| 37 | HXaCHAPTw_CBD2638 CDS | artificial construct |
| 38 | HXaCHAPTw_CBD2638 PRT | artificial construct |
| 39 | HXaCHAPTw_CWT-LST CDS | artificial construct |
| 40 | HXaCHAPTw_CWT-LST PRT | artificial construct |
| 41 | HXaCHAPTw_CWT-NM3 CDS | artificial construct |
| 42 | HXaCHAPTw_CWT-NM3 PRT | artificial construct |
| 43 | HXaM23-LST_CBD2638 CDS | artificial construct |
| 44 | HXaM23-LST_CBD2638 PRT | artificial construct |
| 45 | HXaM23-LST_CWT-LST CDS | artificial construct |
| 46 | HXaM23-LST_CWT-LST PRT | artificial construct |
| 47 | HXaM23-LST_CWT-NM3 CDS | artificial construct |
| 48 | HXaM23-LST_CWT-NM3 PRT | artificial construct |
| 49 | HXaAmi2638_Ami2638_CBD2638 CDS | artificial construct |
| 50 | HXaAmi2638_Ami2638_CBD2638 PRT | artificial construct |
| 51 | HXaAmi2638_Ami2638_CWT-LST CDS | artificial construct |
| 52 | HXaAmi2638_Ami2638_CWT-LST PRT | artificial construct |
| 53 | HXaAmi2638_Ami2638_CWT-NM3 CDS | artificial construct |
| 54 | HXaAmi2638_Ami2638_CWT-NM3 PRT | artificial construct |
| 55 | HXaCHAPK_CHAPK_CBD2638 CDS | artificial construct |
| 56 | HXaCHAPK_CHAPK_CBD2638 PRT | artificial construct |
| 57 | HXaCHAPK_CHAPK_CWT-LST CDS | artificial construct |
| 58 | HXaCHAPK_CHAPK_CWT-LST PRT | artificial construct |
| 59 | HXaCHAPK_CHAPK_CWT-NM3 CDS | artificial construct |
| 60 | HXaCHAPK_CHAPK_CWT-NM3 PRT | artificial construct |
| 61 | HXaCHAPTw_CHAPTw_CBD2638 CDS | artificial construct |
| 62 | HXaCHAPTw_CHAPTw_CBD2638 PRT | artificial construct |
| 63 | HXaCHAPTw_CHAPTw_CWT-LST CDS | artificial construct |
| 64 | HXaCHAPTw_CHAPTw_CWT-LST PRT | artificial construct |
| 65 | HXaCHAPTw_CHAPTw_CWT-NM3 CDS | artificial construct |
| 66 | HXaCHAPTw_CHAPTw_CWT-NM3 PRT | artificial construct |
| 67 | HXaM23-LST_M23-LST_CBD2638 CDS | artificial construct |
| 68 | HXaM23-LST_M23-LST_CBD2638 PRT | artificial construct |
| 69 | HXaM23-LST_M23-LST_CWT-LST CDS | artificial construct |
| 70 | HXaM23-LST_M23-LST_CWT-LST PRT | artificial construct |
| 71 | HXaM23-LST_M23-LST_CWT-NM3 CDS | artificial construct |
| 72 | HXaM23-LST_M23-LST_CWT-NM3 PRT | artificial construct |
| 73 | M23-LST_Ami2638_CBD2638 PRT | artificial construct |
| 74 | M23-LST_Ami2638_CBD2638 PRT* | artificial construct |
| 75 | MART-1 peptide | artificial construct |

Uneven SEQ ID NOs: 1-71 represent the coding sequences (CDS) of even SEQ ID NOs: 2-72 that represent the polypeptide (PRT) sequences.

### FIGURE LEGENDS

FIG. 1. Plate lysis assay with DermiVeil™ (left column), Oat Com™ (middle column) and Oat Silk™ (right column) coated with different amounts of XZ.700 spotted on an S. aureus Newman lawn. The concentrations 1 μg (first row), 10 μg (second row) and 100 μg (third row) XZ.700 per gram powder were tested for each powder. The uncoated powder (fourth row) served as control. A clear lysis zone around the powder can be observed for 100 μg XZ.700 per gram powder.

FIG. 2. Plate lysis assay comparing the three powders DermiVeil™ (first column), Oat Com™ (middle column) and Oat Silk™ (right column) coated with 100 μg XZ.700 per gram powder after heat treatment. First row shows samples stored at room temperature, second row shows samples incubated for 1 h at 120° C., third row shows uncoated powder stored at room temperature, and the last row shows uncoated powder incubated for 1 h at 120° C.

FIG. 3. Plate lysis assay comparing sucrose coated with 100 μg XZ.700 per gram powder (left) after heat treatment with uncoated sucrose (right). First row shows samples stored at room temperature, second row shows samples incubated for 1 h at 100° C., third row shows samples incubated for 1 h at 110° C. and the last row shows samples incubated for 1 h at 120° C.

FIG. 4. Plate lysis assay comparing mannitol coated with 100 μg XZ.700 per gram powder (left) after heat treatment with uncoated sucrose (right). First row shows samples stored at room temperature, second row shows samples incubated for 1 h at 100° C., third row shows samples incubated for 1 h at 110° C. and the last row shows samples incubated for 1 h at 120° C.

FIG. 5. Plate lysis assay comparing starch coated with 100 μg XZ.700 per gram powder (left) after heat treatment with uncoated sucrose (right). First row shows samples stored at room temperature, second row shows samples incubated for 1 h at 100° C., third row shows samples incubated for 1 h at 110° C. and the last row shows samples incubated for 1 h at 120° C.

FIG. 6. Normalized OD_{600 nm} measured over one hour for an enzyme concentration range of 50 nM to 6.25 nM of XZ.700 coated on Oat Com™. XZ.700 kept its lytic potential after exposure of 1 h at up to 130° C. At 135° C. the enzyme was inactivated.

FIG. 7. Normalized OD_{600 nm} measured over one hour for an enzyme concentration range of 50 nM to 6.25 nM of XZ.700 coated on Oat Silk™. XZ.700 kept its lytic potential after exposure of 1 h at up to 120° C. At 130° C. lytic activity was reduced and at 135° C. the enzyme was inactivated.

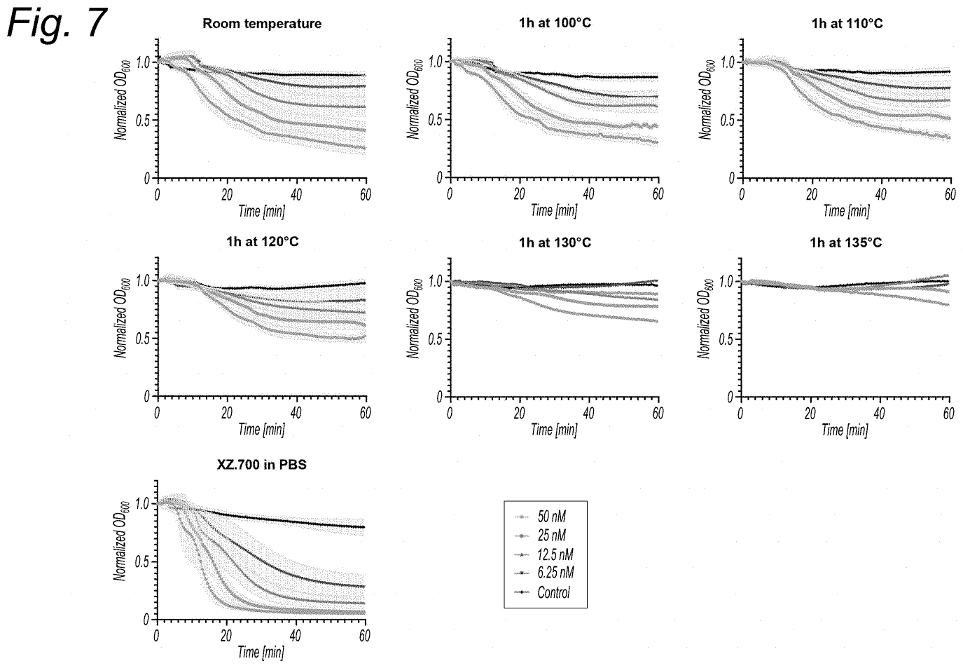

FIG. 8. Normalized OD_{600 nm} measured over one hour for an enzyme concentration range of 50 nM to 6.25 nM of XZ.700 coated on DermiVeil™. No lytic activity of XZ.700 was measured for all temperatures tested. Due to loss of activity even at room temperature the assay was performed only once (no statistical analysis).

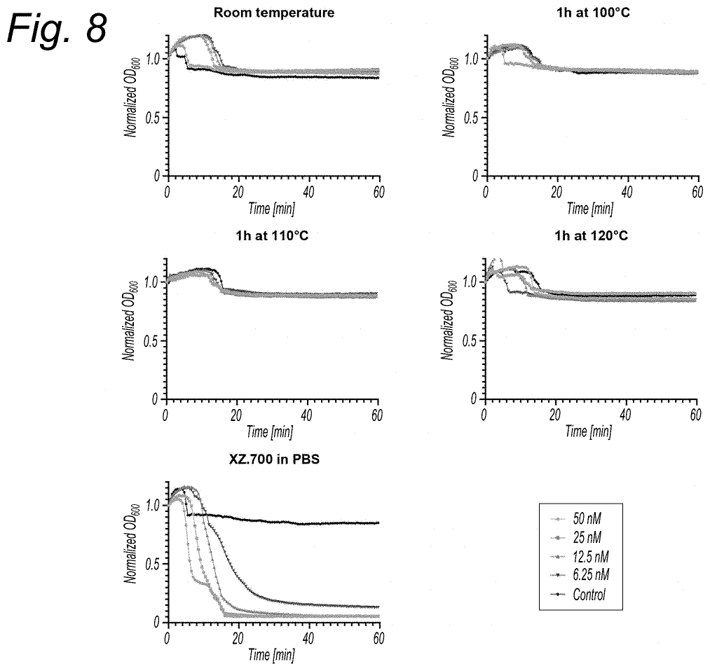

FIG. 9. Normalized OD_{600 nm} measured over one hour for an enzyme concentration range of 50 nM to 6.25 nM of XZ.700 coated on starch. Lysis was observed for samples at room temperature (A), 100° C. (B) and 110° C. (C). At 120° C. (D) lytic activity was lost.

FIG. 10. Normalized OD_{600 nm} measured over one hour for an enzyme concentration of 50 nM of XZ.700 coated on mannitol. Some lytic potential was measured for samples at room temperature (A). The samples exposed to 100° C. (B), 110° C. (C) and 120° C. (D) had lost their lytic activity.

FIG. 11. Normalized OD_{600 nm} measured over one hour for an enzyme concentration of 50 nM of XZ.700 coated on sucrose. Lytic activity was observed for samples at room temperature (A). The samples exposed to 100° C. (B), 110° C. (C) and 120° C. (D) had lost their lytic activity.

FIG. 12. Normalized OD_{600 nm} measured over one hour for an enzyme concentration range of 50 nM to 6.25 nM of HPly511 coated on Oat Com™. HPly511 kept its lytic potential after exposure of 1 h at up to 135° C.

FIG. 13. Normalized OD_{600 nm} measured over one hour for an enzyme concentration range of 50 nM to 6.25 nM of HPly511 coated on Oat Silk™. HPly511 kept its lytic potential after exposure of 1 h at up to 135° C. (F).

FIG. 14. Normalized OD_{600 nm} measured over one hour for an enzyme concentration range of 50 nM to 6.25 nM of HPly511 coated on DermiVeil™. Lytic activity of HPly511 was measured for room temperatures and to some extent for samples exposed to 100° C. for 1 h (B). After 1 h at 110° C. (C) and 120° C. (D) activity of HPly511 was lost.

FIG. 15. Normalized OD_{600 nm} measured over one hour for an enzyme concentration range of 50 nM to 6.25 nM of HPly511 coated on starch. Lysis was observed for samples at room temperature (A), 100° C. (B) and 110° C. (C). At 120° C. (D) the protein was mostly inactivated.

FIG. 16. Normalized OD_{600 nm} measured over one hour for an enzyme concentration of 50 nM of HPly511 coated on mannitol. Some lytic potential was measured for samples at room temperature (A). only minor activity was detected in samples exposed to 100° C. (B). The samples exposed to 110° C. (C) and 120° C. (D) had lost their lytic activity.

FIG. 17. β-Galactosidase coated onto different carriers and spotted on chromogenic coliform agar after exposure to temperatures between 75° C. and 135° C. for 1 h. Uncoated carrier was used as control (right side of the plates). Oat Com™ (A) and Oat Silk™ (B) remained fully active after 1 h at 120° C., but only minor activity was detected after exposure to 135° C. DermiVeil™ (C) showed good activity at room temperature and residual activity at 75° C. and 100° C. β-Galactosidase coated on starch (D) exhibited full activity up to 100° C. and reduced activity at 120° C. Mannitol (E) preserved minor residual activity only at room temperature.

### DEFINITIONS

A bacteriocin herein may be any bacteriocin known to the person skilled in the art, preferably a bacteriocin of any Class I-IV.

Class I bacteriocins herein are small peptide inhibitors and include nisin and other I antibiotics. Class II bacteriocins herein are small (<10 kDa) heat-stable proteins. This class is subdivided into five subclassses. The class IIa bacteriocins (pediocin-like bacteriocins) are the largest subgroup and contain an N-terminal consensus sequence -Tyr-Gly-Asn-Gly-Val-Xaa-Cys across this group. The C-terminal is responsible for species-specific activity, causing cell-leakage by permeabilizing the target cell wall. The class IIb bacteriocins (two-peptide bacteriocins) require two different peptides for activity. One such an example is lactococcin G, which permeabilizes cell membranes for monovalent ions such as Na and K, but not for divalents ones. Almost all of these bacteriocins have a GxxxG motif. This motif is also found in transmembrane proteins where they are involved in helix-helix interactions. The bacteriocin's GxxxG motif can interact with the motifs in the membranes of the bacterial cells and kill the bacteria by doing so. Class IIc encompasses cyclic peptides, which possesses the N-terminal and C-terminal regions covalentely linked. Enterocin AS-48 is the prototype of this group. Class IId cover single-peptide bacteriocins, which are not post-translated modified and do not show the pediocin-like signature. The best example of this group is the highly stable aureocin A53. This bacteriocin is stable under highly acidic environment (HCl 6 N), not affected by proteases and thermoresistant. The most recently proposed subclass is the Class IIe, which encompasses those bacteriocins composed by three or four non-pediocin like peptides. The best example is aureocin A70, a four-peptides bacteriocin, highly active against L. monocytogenes, with potential biotechnological applications.

Class III bacteriocins are large, heat-labile (>10 kDa) protein bacteriocins. This class is subdivided in two subclasses: subclass IIIa or bacteriolysins and subclass IIIb. Subclass IIIa comprises those peptides that kill bacterial cells by cell-wall degradation, thus causing cell lysis. The best studied bacteriolysin is lysostaphin, a 27 kDa peptide that hydrolises several Staphylococcus spp. cell walls, principally S. aureus. Subclass IIIb, in contrast, comprises those peptides that do not cause cell lysis, killing the target cells by disrupting the membrane potential, which causes ATP efflux.

Class IV bacteriocins are defined as complex bacteriocins containing lipid or carbohydrate moities. Confirmatory experimental data was only recently established with the characterization of Sublancin and Glycocin F (GccF) by two independent groups.

A preferred bacteriocin is selected from the group consisting of an acidocin, actagardine, agrocin, alveicin, aureocin, aureocin A53, aureocin A70, carnocin, carnocyclin circularin A, colicin, Curvaticin, divercin, duramycin, Enterocin, enterolysin, epidermin/gallidermin, erwiniocin, gassericin A, glycinecin, halocin, haloduracin, lactocin S, lactococin, lacticin, leucoccin, lysostaphin macedocin, mersacidin, mesentericin, microbisporicin, microcin S, mutacin, nisin, paenibacillin, planosporicin, pediocin, pentocin, plantaricin, pyocin, reutericin 6, sakacin, salivaricin, subtilin, sulfolobicin, thuricin 17, trifolitoxin, variacin, vibriocin, warnericin and a warnerin.

The bacteriocin may be from a bacterium itself (24), such as, but not limited to a pyocin from Pseudomonas aeruginosa, preferably pyocin SA189 (25).

The antimicrobial peptide may be any antimicrobial peptide known to the person skilled in the art. Sometimes in the art, antimicrobial peptides are considered bacteriocins as listed here above. A preferred antimicrobial peptide is selected from the group consisting of a cationic or polycationic peptide, an amphipatic peptide, a sushi peptide, a defensin and a hydrophobic peptide.

The bacterial autolysin may be any a bacterial autolysin known to the persons killed in the art. A preferred bacterial autolysin is LytM. An antibacterial protein may be lactoferrin or transferrin. A bacteriophage endolysin may or may not be comprised in a bacteriophage.

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence. A “nucleic acid construct” is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for the polypeptide of the invention such that the control sequence directs the production/expression of the peptide or polypeptide of the invention in a cell and/or in a subject. “Operably linked” may also be used for defining a configuration in which a sequence is appropriately placed at a position relative to another sequence coding for a functional domain such that a chimeric polypeptide is encoded in a cell and/or in a subject.

“Expression” is construed as to include any step involved in the production of the peptide or polypeptide including, but not limited to, transcription, post-transcriptional modification, translation, post-translational modification and secretion.

A “control sequence” is defined herein to include all components which are necessary or advantageous for the expression of a polypeptide. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended in the present invention, the term usually refers to an extrachromosomal, self-replicating vector which harbors a selectable antibiotic resistance.

An “expression vector” may be any vector which can be conveniently subjected to recombinant DNA procedures and can bring about the expression of a nucleotide sequence encoding a polypeptide of the invention in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide −1 of the transcription start site (TSS).

A “polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 10% of the value.

All patent and literature references cited in the present specification are hereby incorporated by reference in their entirety.

The following examples are offered for illustrative purposes only, and are not intended to limit the scope of the present invention in any way.

### Examples

### Introduction

Endolysins are phage derived peptidoglycan hydrolases produced at the end of the lytic cycle to release progeny virions (Schmelcher, Donovan et al. 2012). They are promising antimicrobials due to their hosts specificity and activity against drug resistant strains. But degradation of proteins and loss of activity in aqueous solution represents a burden for protein therapeutics (Manning, Patel et al. 1989). The chimeric endolysin XZ.700 shows potent lytic activity against Staphylococcus aureus but loss of activity over time is observed in aqueous solutions. Therefore, bringing the protein of interest into a solid state through lyophilisation could increase protein stability. In order to control therapeutic dose and enable XZ.700 application on skin, a carrier for the lyophilized protein is needed.

Colloidal oatmeal was declared a safe ingredient for dermal application by the Food and Drug Administration (FDA) in 1989 (Fowler 2014). Due to its anti-inflammatory characteristic, colloidal oatmeal is used to treat different skin conditions including atopic dermatitis (Fowler 2014). These beneficial features render colloidal oatmeal a promising carrier. The two oatmeal derived powders (Avena sativa) Oat Com™ and Oat Silk™ from Oat Cosmetics (The University of Southampton Science Park, 2 Venture Road, Chilworth, Southampton, Hampshire, SO16 7NP, United Kingdom) were selected as carriers. Additionally, the barley starch powder DermiVeil™ (Hordeum vulgare), mannitol, sucrose and starch were included as potential carriers.

In order to test whether carrier coating and lyophilisation also increases stability of other proteins, different enzymatic active proteins were included in the study. The Listeria phage endolysin HPly511 (Eugster and Loessner 2012) containing a His-tag for purification was included to proof the concept for endolysins in general. The activity of the two endolysins was evaluated in different lytic assays. Luciferase, β-Galactosidase and horseradish peroxidase (HRP) were selected due to their simple activity detection with luminescence measurements or colorimetric assays. The firefly luciferase from Photinus pyralis and the β-Galactosidase were purchased as lyophilized powders. Luciferase activity could be detected as light generated during a two-step reaction catalyzed by the enzyme. β-Galactosidase activity was measured in a colorimetric assay. Hydrolysis of the compound Salmon-β-D-galactosidase leads to a red stain indicating preserved activity of the enzyme. The horseradish peroxidase used in this study was fused to the Salmonella S16 bacteriophage long tail fiber (LTF) and provided by Matthew Dunne (Foodmicrobiology Lab, ETH Zurich). The LTF-HRP conjugation product was developed for rapid Salmonella detection (Denyes, Dunne et al. 2017). Oxidation of 3,3′,5,5′-Tetramethylbenzidine leads to the formation of a blue diamine, which can be measured and reflects the remaining activity of HRP.

### Materials and Methods

### Materials: Media, Buffers and Carriers

Growth media (Table 2) and all buffers (Table 3) except when used for dialysis were autoclaved at 121° C. for 20 min. Carriers (Table 4) came as dry powders and were used directly.

TABLE 2
Growth media used for activity assays.

| | Composition | | | | |
|---|---|---|---|---|---|
| | | Mass/volume | | | Catalog |
| Media | Ingredients | per L | pH | Supplier | Number |
| TSB ^{1) 2)} | Tryptic soy broth | 30 g | 7.3 | Biolife | 4021552 |
| ½ BHI ¹⁾ | Brain heart | 18.5 g | 7.4 | Biolife | 4012302 |
| | infusion broth | | | | |
| Chromo | Chromogenic | 27.1 g | 6.8 | Biolife | 4012972 |
| Agar ¹⁾ | coliform agar | | | | |
| | iso formulation | | | | |

¹⁾ PURELAB ELGA water (Labtech) to fill up to desired final volume
²⁾ for agar plates 12 g/L Agar Agar Kolbe I (Roth; Catalog number: 5210.5) was added

TABLE 3
Buffers used for carrier coating and activity assays.

| | Composition | | | | | |
|---|---|---|---|---|---|---|
| | | Mass/volume | | | | Catalog |
| Buffer | Ingredients | per L | | pH | Supplier | Number |
| 20 mM Tris | Tris Base | 2.42 | g | 7.4 | Sigma-Aldrich | 11814273001 |
| buffer ^{1) 2)} | | | | | | |
| 50 mM Tris | Tris Base | 6.05 | g | 7.4 | Sigma-Aldrich | 11814273001 |
| buffer ^{1) 2)} | | | | | | |
| 1M Tris | Tris Base | 121.14 | g | 7.8 | Sigma-Aldrich | 11814273001 |
| buffer ^{1) 2)} | | | | | | |
| PBS ¹⁾ | 120 mM NaCl | 7.01 | g | 7.4 | Fisher Scientific | 1000152 |
| | 50 mM | 17.91 | g | | Sigma-Aldrich | 71650-1KG |
| | Na₂HPO₄•12H₂O | | | | | |

¹⁾ PURELAB Chorus ELGA water (18.2 MΩcm; Labtech) to fill up to desired final volume
²⁾ pH was adjusted using 0.1-10M NaOH (Merck) or 0.1-4M HCl (Sigma-Aldrich)

TABLE 4
Carriers used for protein coating.

| Carrier | Supplier | Catalog Number |
|---|---|---|
| Oat Com ™ | Oat cosmetics | A0839 |
| Oat Silk ™ | Oat cosmetics | T7660-25G |
| DermiVeil ™ | Oat cosmetics | T832.2 |
| d-Mannitol | Sigma-Aldrich | 63560-250G-F |
| Starch | Merck | 101252 |
| Sucrose | Roth | 9286.1 |

### Methods

### Protein Coating on Powders

Two oat meal derived powders, Oat Com™ and Oat Silk™, the barley powder DermiVeil™, mannitol, sucrose and starch were used as carriers (Table 4) and coated with different proteins (Table 5). First trials were performed with Oat Com™, Oat Silk™ and DermiVeil™ coated with either 1 μg, 10 μg or 100 μg XZ.700 per gram powder. The other carriers were coated with 100 μg XZ.700 per gram powder. In brief, 1 g of each type was weighted and ultrapure water (18.2 MΩcm, Labtech) was used to make a suspension. The volumes were adjusted to the different powder types (Table 5). XZ.700 and HPly511 were dialyzed against 20 mM Tris Buffer (Table 3) in a Spectra/Pore dialysis tubing (6-8 kD molecular weight cut off, Sprectrum Laboratories) over night. Lyophilized luciferase from Photinus pyralis (SigmaAldrich; Catalog number: SRE0045-2MG) was resuspended in 1M Tris buffer (Table 3) and then dialyzed against 50 mM Tris Buffer (Table 3) in a Spectra/Pore dialysis tubing (6-8 kD molecular weight cut off, Spectrum Laboratories) over night. Lyophilized β-Galactosidase (Sigma Aldrich; Catalog number: 48275-1MG-F) was directly resuspended in 20 mM Tris buffer (Table 3). The S16 long tail fiber with horseradish peroxidase conjugated onto it was synthesized according to state of the art techniques. Protein concentrations were determined by absorption measurement at 280 nm (A280, Nanodrop) and values were corrected by the theoretical absorption coefficient of the proteins calculated with CLCBio software. The proteins were then added to the suspensions. The mixtures were frozen at −80° C. prior to lyophilization (−46° C., vacuum 211 μB, condenser −45.6). The lyophilized products were stored dry at room temperature.

TABLE 5
Volumes needed to resuspend different powder types and the amount
of protein added to the suspensions prior to lyophilisation.

| | | Ultrapure | | | | Firefly | β- |
|---|---|---|---|---|---|---|---|
| | Mass | water | XZ.700 | HPLY511 | LTF-HRP | luciferase | Galactosidase |
| Powder | [g] | [mL] | [μg] | [μg] | [μg] | [μg] | [μg] |
| DermiVeil | 1 | 1 | 1, 10, 100 | 100 | 100 | 100 | 100 |
| Oat Com | 1 | 9 | 1, 10, 100 | 100 | 100 | 100 | 100 |
| Oat Silk | 1 | 4 | 1, 10, 100 | 100 | 100 | 100 | 100 |
| Mannitol | 1 | 1 | 100 | 100 | 100 | 100 | 100 |
| Starch | 1 | 2 | 100 | 100 | 100 | 100 | 100 |
| Sucrose | 1 | 1 | 100 | — | — | — | — |

### Plate Lysis Assay

In order to test the activity of XZ.700 after the coating process, the samples were spotted on a square TSA plate (Table 2) containing S. aureus Newman (Staphylococcus aureus subsp. aureus Rosenbach, ATCC® 25904 ™). S. aureus Newman was grown to an OD_{600 nm} between 0.4 and 0.6 in TSB (Table 2) and 5 mL of the culture were spread on the plate. Excess liquid was discarded and the plate was dried in a laminar flow hood for 15 min. 5 mg of powder was spotted onto the plate using a spatula. The plate was then incubated over night at 30° C.

To assess the heat stability of XZ.700 coated onto solid supports (powders), the samples were incubated for 1 h in a PCR gradient thermocycler at temperatures between 50° C. and 100° C. Additionally, samples were exposed to 100° C., 110° C. and 120° C. for 1 h and to 100° C. for 24 h in a heat block. Oat Com™ samples were additionally exposed to 130° C. for 1 h. All samples were spotted on a TSA plates as described above.

### Turbidity Reduction Assay

Activity of XZ.700 and HPly511 was tested after reconstitution in PBS as the drop of optical density over time. S. aureus Newman for XZ.700 and L. monocytogene 1001 for HPly511 were cultured in ½ BHI medium (Table 2) to an OD_{600 nm} of 0.4. The cells were then harvested at 7000 g (at 4° C. for 10 min; Beckman Coulter, JA-10 Rotor) and washed with PBS (Table 3). The pellet was resuspended in 1% of the original culture volume PBS (Table 3) and 200 μL aliquots were stored at −80° C. until use.

1 mL PBS (Table 3) was added to 52 mg powder with XZ.700 and 37.8 mg powder with HPly511 (coated with 100 μg endolysin per g powder) in order to obtain a theoretical protein concentration of 100 nM. The suspensions were incubated at 4° C. in an overhead rotator at 10 rpm for 2 h and then centrifuged at 30000 g for 30 min at 4° C. to obtain a clear solution (Sigma 3K 30, 19777 rotor). The supernatant was used to prepare a two-fold dilution series in PBS (Table 3) on a 96 well plate leading to concentrations between 50 nM and 6.25 nM. The corresponding substrate cells were diluted in PBS (Table 3) to an OD_{600 nm} of 2.0 leading to an OD_{600 nm} of 1.0 on the 96 well plate at time point zero. The OD_{600 nm} was measured every 30 s for one hour using an Omega Photospectrometer (FLUOstaa Omega, BMG LABTECH). The values were normalized and used to plot the lysis curve. The same procedure was done with heat treated samples (exposed to 100° C., 110° C., 120° C., 130° C. and 135° C. for 1 h) to test heat stability of the protein on different carriers.

### LTF-HRP Colorimetric Assay

Activity of LTF-HRP was tested after reconstitution in PBS (Table 3). Ten mg of powder coated with luciferase and its uncoated control were weighted and heat treated (room temperature, 75° C., 100° C., 125° C. 135° C. for 1 h). 1 mL PBS (Table 3) was added to the samples in order to obtain a theoretical protein concentration of 1 μg/mL. The suspensions were incubated at 4° C. in an overhead rotator at 10 rpm for 2 h and then centrifuged at 30000 g for 30 min at 4° C. to obtain a clear solution (Sigma 3K 30, 19777 rotor). 99 μL of TMB solution (Merck; Catalog number. 613544-100ML) was pipetted per well of a 96-well plate and then 1 μL of the supernatant was added. A LTF-HRP stock served as positive control (2 μg/mL in PBS). Oxidation of 3,3′,5,5′-Tetramethylbenzidine leads to the formation of a blue diamine which can be measured as absorbance at 370 nm reflecting the remaining activity of HRP. The absorbance was measured after 15 min in an Omega Photospectrometer (FLUOstaa Omega, BMG LABTECH). Thresholds were defined to categorize remaining activity (x<0.1 no activity, 0.1≤x<1.0 some residual activity and x≥1.0 activity preserved).

### Luciferase Glow Assay

Activity of the firefly luciferase was tested after reconstitution in 1M Tris buffer (Table 3). 24.8 mg of powder coated with luciferase and its uncoated control were weighted and heat treated (room temperature, 75° C., 100° C., 125° C. 135° C. for 1 h). 400 μL 1M Tris buffer (Table 3) was added to the samples in order to obtain a theoretical protein concentration of 100 nM. The suspensions were incubated at 4° C. in an overhead rotator at 10 rpm for 2 h and then centrifuged at 30000 g for 30 min at 4° C. to obtain a clear solution (Sigma 3K 30, 19777 rotor). 25 μL of the samples were distributed on a white 96-well plate. 100× d-luciferin from the Pierce™ Firefly Luciferase Glow Assay Kit (ThermoFisher Scientific; Catalog number: 16176) was diluted in glow assay buffer. This reaction mix was added to the samples in order to obtain a 1:1 ratio. A 400 nM luciferase stock solution was used as positive control, the uncoated powder suspensions served as negative control and 1M Tris buffer (Table 3) was used as blank. Luminescence was measured in a GloMax® Navigator (Promega) after keeping the plate for 10 min in the dark inside the machine. All measured values were corrected by subtracting the blank. In order to exclude background luminescence of the carriers, the value of the corresponding uncoated control was subtracted from the sample values. Thresholds were defined to categorize remaining activity (x<10² no activity, 10²≤x<10⁴ some residual activity and x≥10⁴ activity preserved).

### β-Galactosidase Colorimetric Assay

In order to test activity of the β-galactosidase after the coating process, samples were spotted on chromogenic coliform agar (Table 2). 5 mg of powder coated with β-galactosidase and its uncoated control was distributed into Eppendorf tubes and heat treated for one hour at different temperatures (room temperature, 75° C., 100° C., 125° C. 135° C.). All samples from the same carrier were spotted on to the same chromo agar plate and kept at room temperature overnight. Color change of the plates at the spot site was used as activity indicator.

### Results

### Plate Lysis Assay

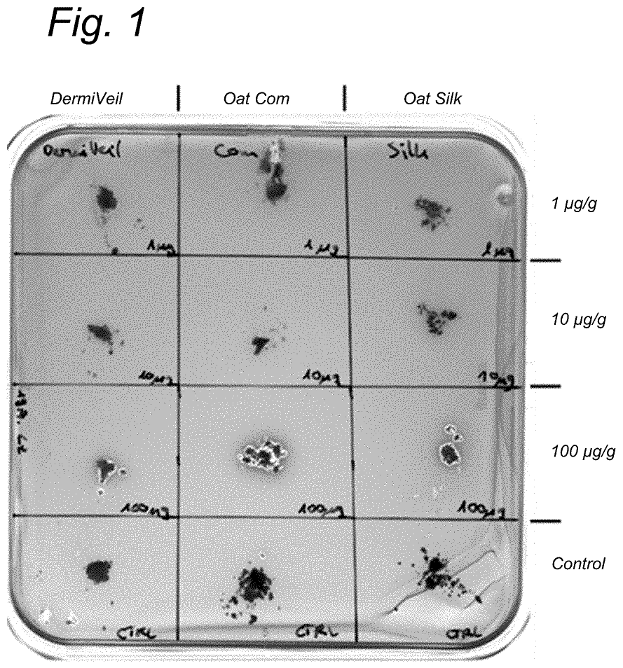

The plate lysis assay in which three different protein concentrations and three different powders (Oat Com™, Oat Silk™, and DermiVeil™) were tested showed clear lysis for 100 μg XZ.700 per gram powder for all three powders (FIG. 1). A concentration of 1 μg or 10 μg XZ.700 per gram powder was too low to result in lysis.

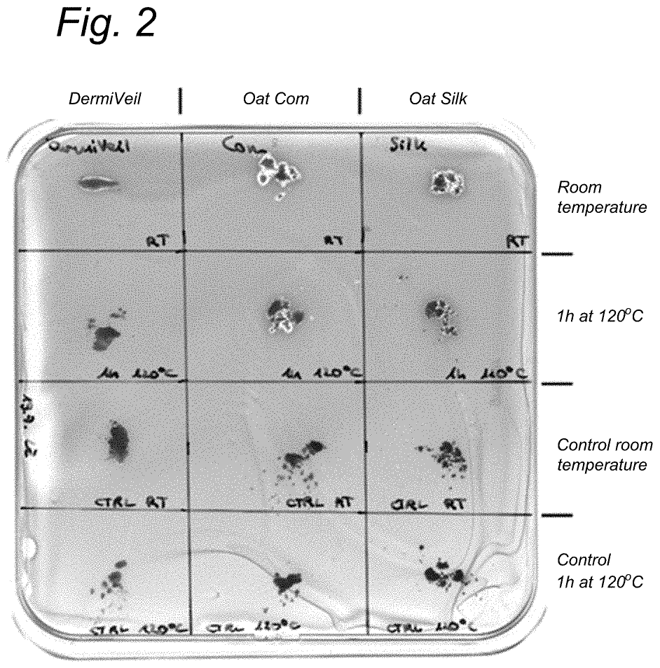

When the samples were exposed to temperature between 50° C. and 100° C. for one hour in a thermocycler, all samples retained their lytic potential (results not shown). The samples heated at 100° C. for one hour in a heat block were still active, whereas at 110° C. XZ.700 coated on DermiVeil™ had lost its activity. After 1 h at 120° C. activity could still be observed for Oat Com™. It seems that for Oat Silk™, only very little residual activity indicated by very small lysis zones is present after heating to 120° C. for 1 h (FIG. 2). Heat treatment for 24 h at 100° C. inactivated the protein in all samples (data not shown).

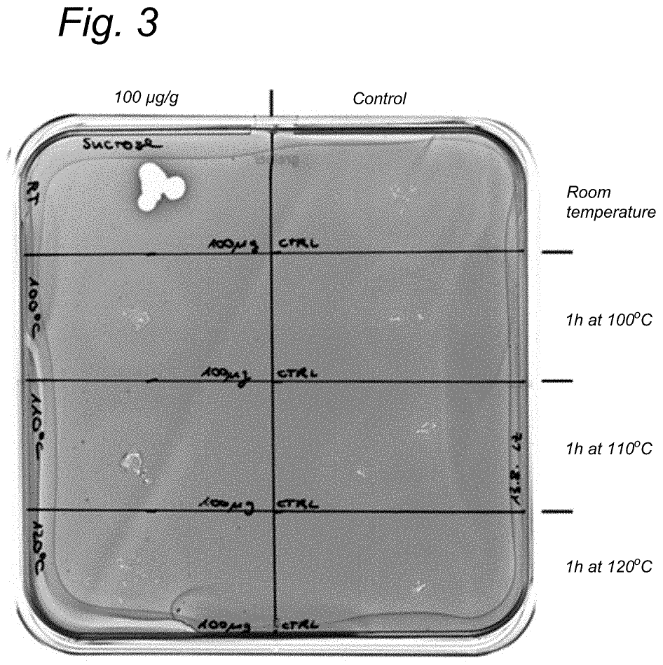

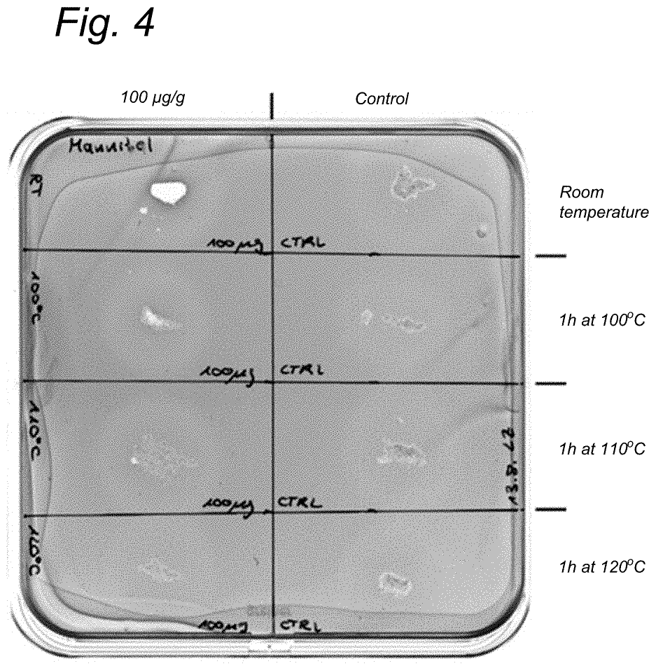

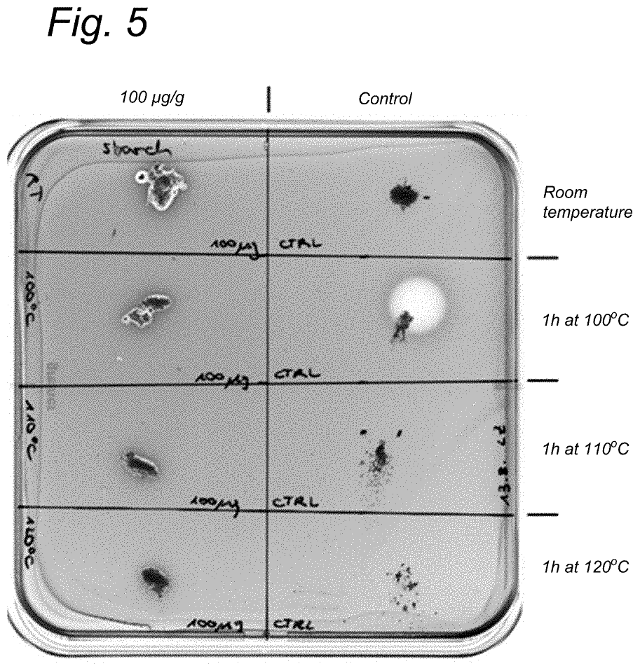

The other carrier materials sucrose, mannitol, starch were exposed to 100° C., 110° C. and 120° C. for one hour prior to spotting on a TSA plate covered with S. aureus Newman. All coated carriers kept at room temperature showed lytic activity. XZ.700 coated on sucrose already lost its activity when exposed to 100° C. for 1 h (FIG. 3). The mannitol samples seem mostly inactivated at 100° C. for 1 h with only minor residual activity being detectable (FIG. 4). A clear lysis zone was visible for the starch samples at room temperature and 100° C., whereas major inactivation with only faint detectable lysis took place at 110° C. (FIG. 5).

XZ.700 coated on Oat Com™ showed highest activity at high temperatures in all replicates (Table 6). The activity of XZ.700 coated on DermiVeil™ seemed to be very unstable over time, as activity was only observed in the first replicate.

TABLE 6
Summary table indicating activity of XZ.700 coated on different carriers
and exposed to temperatures between 100° C. and 130° C. for
1 h. The activity is coded: yes = clear lysis zone; some =
small lysis zone; no = no lysis; not tested = temperature was not tested for this carrier.

| | Replicate 1 | | | | | Replicate 2 | | | | |
|---|---|---|---|---|---|---|---|---|---|---|
| Carrier | RT | 100° C. | 110° C. | 120° C. | 130° C. | RT | 100° C. | 110° C. | 120° C. | 130° C. |
| Oat Com | yes | yes | yes | yes | not | yes | yes | yes | yes | no |
| | | | | | tested | | | | | |
| Oat Silk | yes | yes | yes | some | not | yes | yes | yes | some | not |
| | | | | | tested | | | | | tested |
| DermiVeil | yes | some | no | no | not | no | no | no | no | not |
| | | | | | tested | | | | | tested |
| Sucrose | yes | no | no | no | not | yes | no | no | no | not |
| | | | | | tested | | | | | tested |
| Mannitol | yes | some | no | no | not | yes | yes | no | no | not |
| | | | | | tested | | | | | tested |
| Starch | yes | yes | yes | some | not | yes | yes | some | no | not |
| | | | | | tested | | | | | tested |

Replicate 3

| Carrier | RT | 100° C. | 110° C. | 120° C. | 130° C. |
|---|---|---|---|---|---|
| Oat Com | yes | yes | yes | yes | no |
| Oat Silk | yes | yes | yes | some | not |
| | | | | | tested |
| DermiVeil | no | no | no | no | not |
| | | | | | tested |
| Sucrose | yes | no | no | no | not |
| | | | | | tested |
| Mannitol | yes | yes | no | no | not |
| | | | | | tested |
| Starch | yes | some | some | some | not |
| | | | | | tested |

### Turbidity Reduction Assay: XZ.700

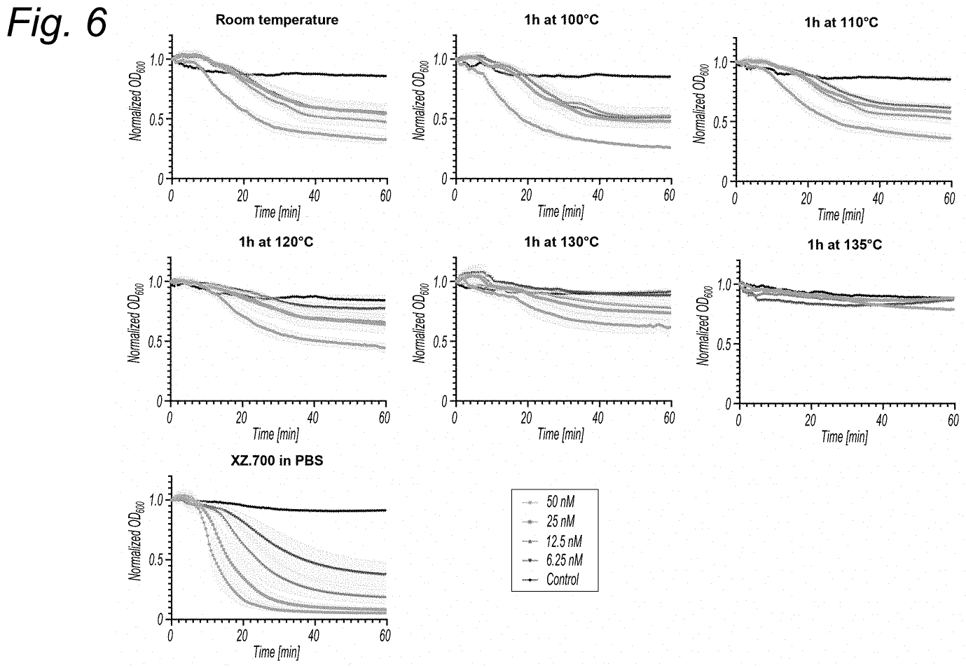

52 mg of powder after reconstitution was used to measure remaining activity reflected in cell lysis. Measuring the drop in optical density of a S. aureus Newman cell suspension during one hour showed activity for Oat Com™ and Oat Silk™, but no activity for DermiVeil™ (FIG. 6A, 7A, 8A). Due to residual powder particles resulting in residual turbidity, fluctuations in the OD_{600 nm} measurements were observed.

The same procedure was applied to samples previously heated for one hour at 100° C., 110° C. and 120° C. in order to test heat stability of XZ.700 on the different carriers. All coated carriers (except for DermiVeil™) showed at least some activity at room temperature. XZ.700 coated on Oat Com™ and Oat Silk™ kept its lytic potential even after exposure to 120° C. (FIG. 6D, 7D). Activity of XZ.700 coated on Oat Com™ and Oat Silk™ was lost after 1 h at 135° C. (FIG. 6F, 7F), whereas 130° C. was not sufficient to inactivate XZ.700 completely (FIG. 6E, 7E).

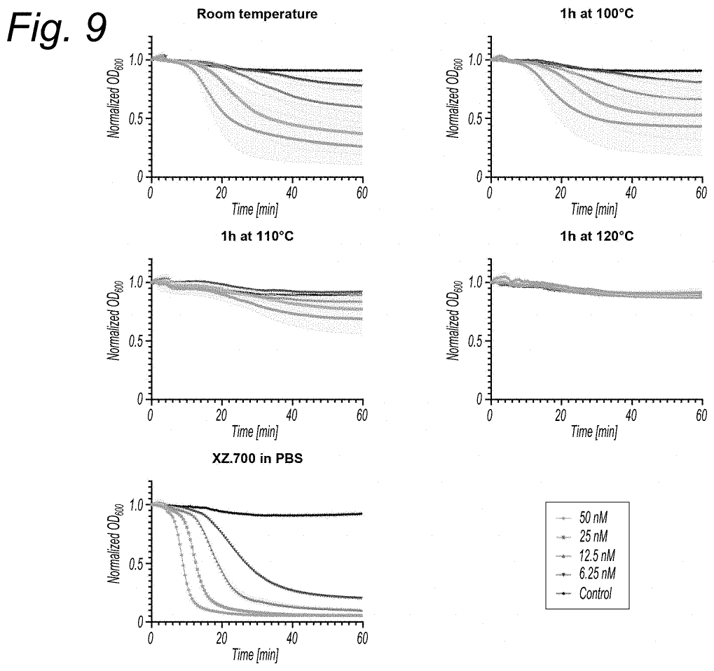

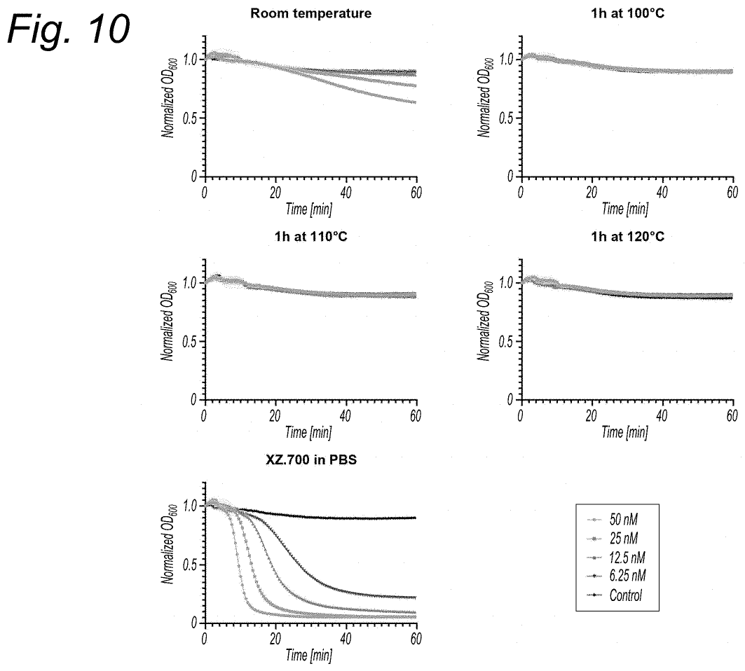

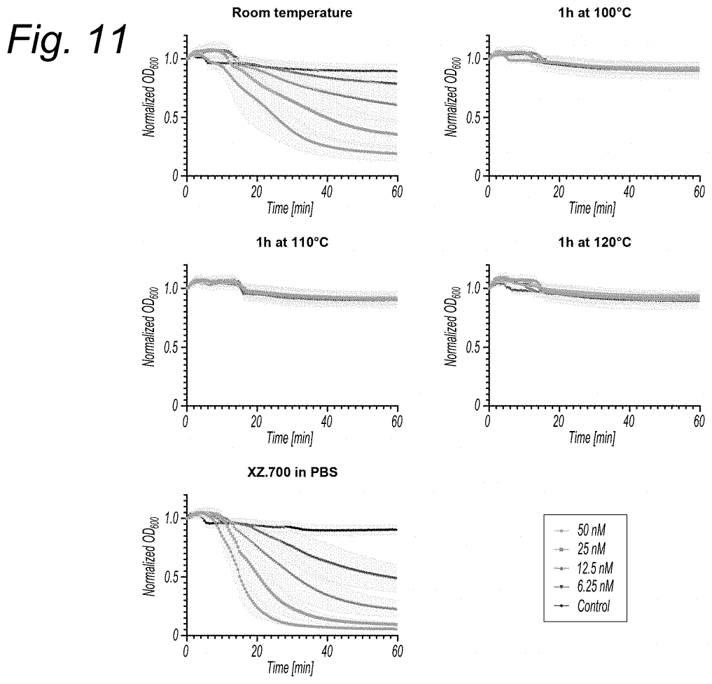

After reconstitution, XZ.700 coated on sucrose, mannitol and starch showed activity for samples stored at room temperature. However, mannitol seems an inferior carrier since it does not fully support XZ.700 activity (FIG. 10A). When coated on starch, XZ.700 was still active after incubation for one hour at 100° C. (FIG. 9). Virtually no activity was detected after 110° C. exposure, and at 120° C. the activity of XZ.700 coated on starch was fully lost. In contrast, XZ.700 coated on mannitol (FIG. 10) or sucrose (FIG. 11) lost its lytic activity already when exposed to 100° C.

Lytic activity of XZ.700 coated onto different carriers and exposed to high temperatures is summarized for each biological replicate in Table 7. Replicate 4 was performed to determine complete heat inactivation.

TABLE 7
Summary table indicating activity of XZ.700 coated on different
carriers and exposed to temperatures between 100°
C. and 135° C. for 1 h. The activity is color-coded:
green = clear lysis curve; blue = some lysis;
red = no lysis; grey = temperature was not tested for this carrier.

| | Replicate 1 | | | | | |
|---|---|---|---|---|---|---|
| Carrier | RT | 100° C. | 110° C. | 120° C. | 130° C. | 135° C. |
| Oat Com | yes | not | yes | yes | not | not |
| | | tested | | | tested | tested |
| Oat Silk | yes | not | yes | yes | not | not |
| | | tested | | | tested | tested |
| DermiVeil | no | not | not | not | not | not |
| | | tested | tested | tested | tested | tested |
| Sucrose | yes | no | no | no | not | not |
| | | | | | tested | tested |
| Mannitol | very | no | no | no | not | not |
| | little | | | | tested | tested |
| Starch | yes | yes | no | no | not | not |
| | | | | | tested | tested |
| | Replicate 2 | | | | | |
| Carrier | RT | 100° C. | 110° C. | 120° C. | 130° C. | 135° C. |
| Oat Com | yes | yes | yes | yes | very | not |
| | | | | | little | tested |
| Oat Silk | yes | yes | yes | yes | not | not |
| | | | | | tested | tested |
| DermiVeil | no | no | no | no | not | not |
| | | | | | tested | tested |
| Sucrose | yes | no | no | no | not | not |
| | | | | | tested | tested |
| Mannitol | some | no | no | no | not | not |
| | | | | | tested | tested |
| Starch | some | no | no | no | not | not |
| | | | | | tested | tested |
| | Replicate 3 | | | | | |
| Carrier | RT | 100° C. | 110° C. | 120° C. | 130° C. | 135 C. |
| Oat Com | yes | yes | yes | yes | yes | not |
| | | | | | | tested |
| Oat Silk | yes | yes | yes | yes | not | not |
| | | | | | tested | tested |
| DermiVeil | not | not | not | not | not | not |
| | tested | tested | tested | tested | tested | tested |
| Sucrose | yes | no | no | no | not | not |
| | | | | | tested | tested |
| Mannitol | very | no | no | no | not | not |
| | little | | | | tested | tested |
| Starch | yes | yes | yes | no | not | not |
| | | | | | tested | tested |
| | Replicate 4 | | | | | |
| Carrier | RT | 100° C. | 110° C. | 120° C. | 130° C. | 135° C. |
| Oat Com | yes | yes | yes | yes | very | no |
| | | | | | little | |
| Oat Silk | yes | yes | yes | yes | some | very |
| | | | | | | very |
| | | | | | | little |
| DermiVeil | not | not | not | not | not | not |
| | tested | tested | tested | tested | tested | tested |
| Sucrose | not | not | not | not | not | not |
| | tested | tested | tested | tested | tested | tested |
| Mannitol | not | not | not | not | not | not |
| | tested | tested | tested | tested | tested | tested |
| Starch | not | not | not | not | not | not |
| | tested | tested | tested | tested | tested | tested |

### Turbidity Reduction Assay: HPIy511

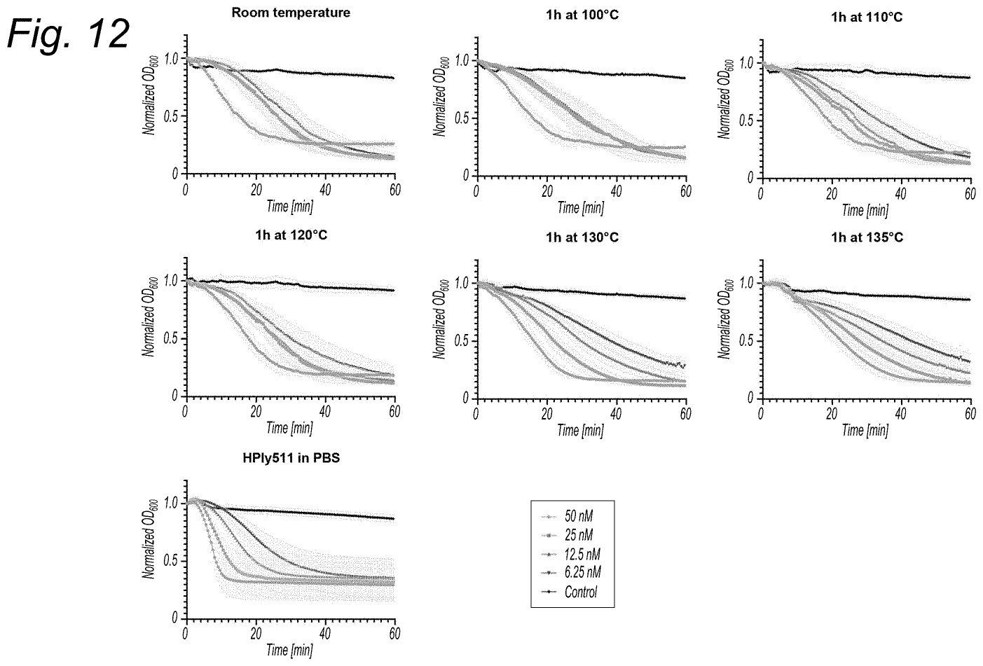

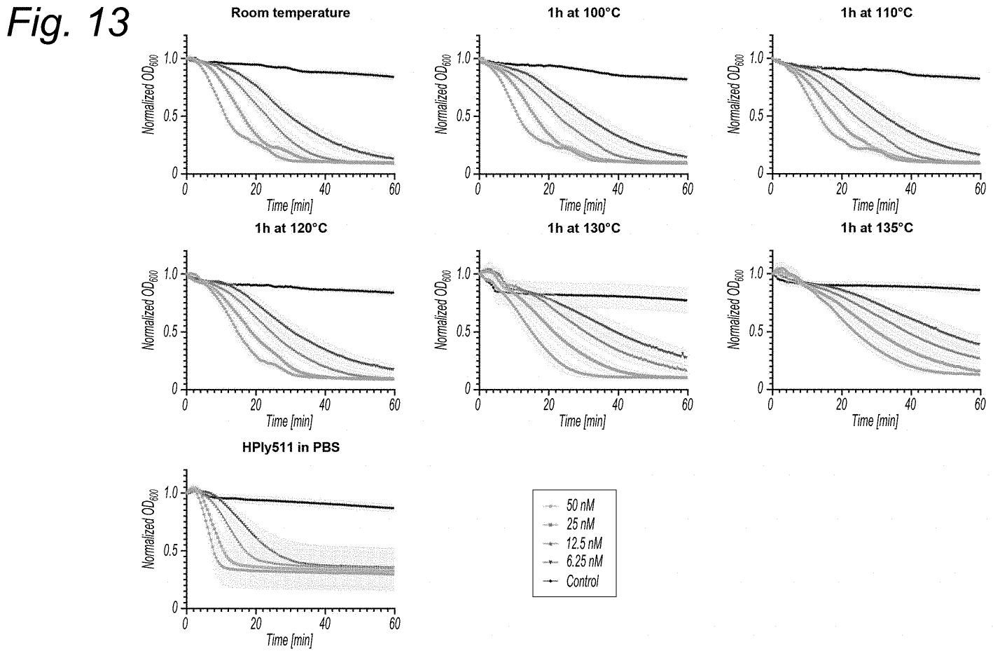

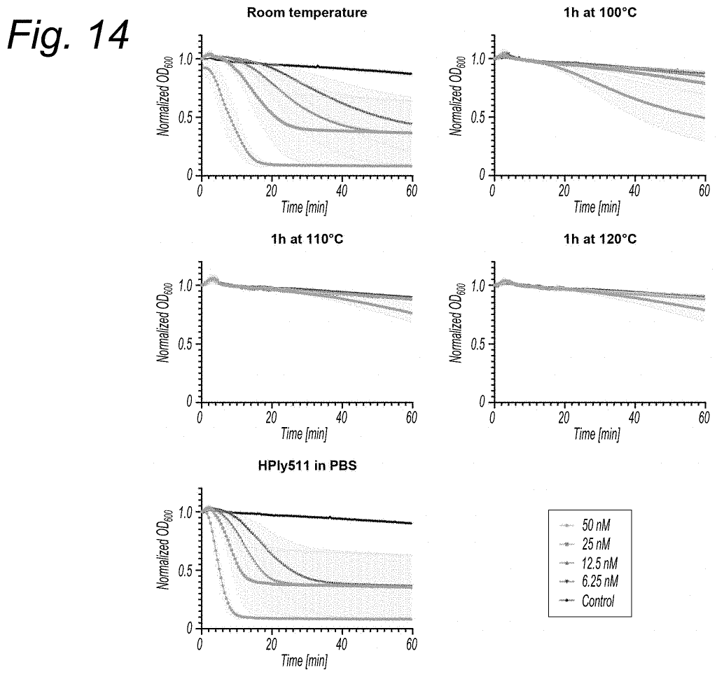

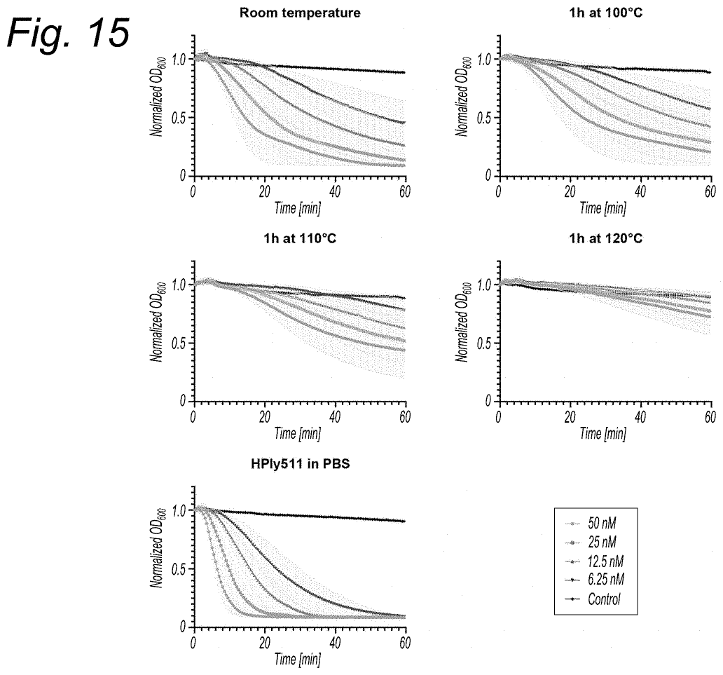

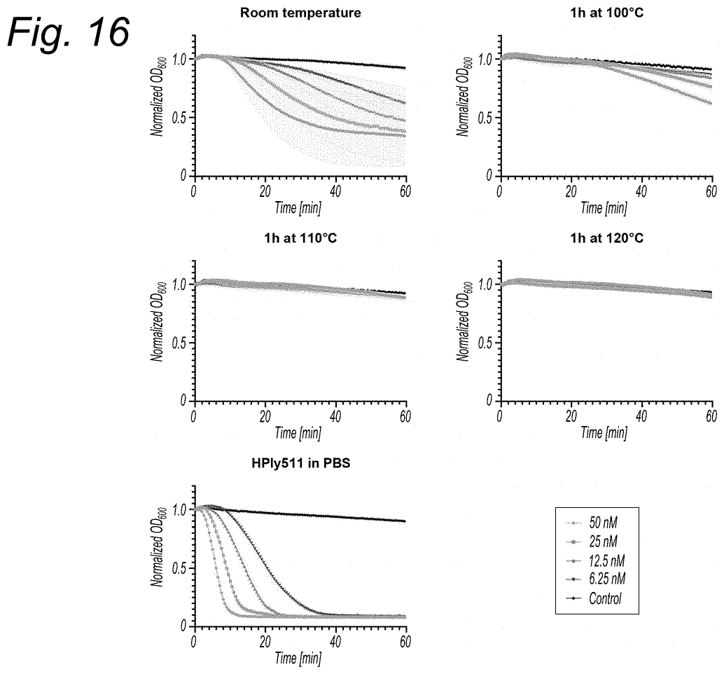

The same procedure as for XZ.700 was applied to test activity of HPly511 coated on different carriers. The drop in optical density of Listeria monocytogenes 1001 substrate cells over one hour showed lytic activity for all carriers at room temperature (FIG. 12A, 13A, 14A, 15A, 16A). Except for mannitol which showed only very little remaining activity after 1 h at 100° C. (FIG. 16B), all other carriers retained lytic activity. For HPly511 coated on DermiVeil™ activity was lost after exposure to 110° C. (FIG. 14C). Only minor activity remained for HPly511 coated on starch (FIG. 15). HPly511 coated on Oat Com™ and Oat Silk™ stayed fully active even after exposure to 135° C. for one hour (FIG. 12F, FIG. 13F).

Lytic activity of HPly511 coated onto different carriers and exposed to high temperatures is summarized for each biological replicate in Table 8.

TABLE 8
Summary table indicating activity of HPly511 coated on different carriers and exposed to temperatures
between 100° C. and 135° C. for 1 h. The activity is coded: yes = clear lysis
curve; some = some lysis; no = no lysis; not tested = temperature was not tested for this carrier.

| | Replicate 1 | | | | | | Replicate 2 | | | | | |
|---|---|---|---|---|---|---|---|---|---|---|---|---|
| Carrier | RT | 100° C. | 110° C. | 120° C. | 130° C. | 135° C. | RT | 100° C. | 110° C. | 120° C. | 130° C. | 135° C. |
| Oat Com | yes | yes | yes | yes | yes | yes | yes | yes | yes | yes | yes | yes |
| Oat Silk | yes | yes | yes | yes | yes | yes | yes | yes | yes | yes | yes | yes |
| DermiVeil | yes | yes | some | some | not | not | yes | some | no | no | not | not |
| | | | | | tested | tested | | | | | tested | tested |
| Mannitol | yes | very | no | no | not | not | yes | some | no | no | not | not |
| | | little | | | tested | tested | | | | | tested | tested |
| Starch | yes | yes | yes | yes | not | not | yes | yes | yes | no | not | not |
| | | | | | tested | tested | | | | | tested | tested |

Replicate 3

| Carrier | RT | 100° C. | 110° C. | 120° C. | 130° C. | 135° C. |
|---|---|---|---|---|---|---|
| Oat Com | yes | yes | yes | yes | yes | yes |
| Oat Silk | yes | yes | yes | yes | yes | yes |
| DermiVeil | yes | no | no | no | not | not |
| | | | | | tested | tested |
| Mannitol | no | very | no | no | not | not |
| | | little | | | tested | tested |
| Starch | yes | some | no | no | not | not |
| | | | | | tested | tested |

### LTF-HRP Colorimetric Assay

Ten mg of LTF-HRP coated powder was reconstituted and remaining activity tested in a colorimetric assay. Table 9 summarizes the color change for each condition in all three replicates (raw data in Appendix, Table 11). Similar to previous assays Oat Com™ and Oat Silk™ retained activity of the coated protein at higher temperatures than the other carriers. HRP coated on DermiVeil™ showed only little activity at room temperature and seemed to be unstable over time. In this setup, starch was much less effective in activity preservation during dry heat exposure than in previous assays coated with other proteins.

TABLE 9
Summary table indicating activity of the horseradish peroxidase coupled to a long tail fiber coated
on different carriers and exposed to temperatures between 75° C. and 135° C. for 1 h.
The activity is coded: yes = clear color change; some = some faint color change; no = no color change.

| | Replicate 1 | | | | | Replicate 2 | | | | |
|---|---|---|---|---|---|---|---|---|---|---|
| Carrier | RT | 75° C. | 100° C. | 120° C. | 135° C. | RT | 75° C. | 100° C. | 120° C. | 135° C. |
| Oat Com | yes | yes | yes | some | some | yes | yes | some | some | no |
| Oat Silk | yes | yes | yes | yes | some | yes | yes | yes | some | some |
| DermiVeil | some | some | no | no | no | some | no | no | no | no |
| Starch | yes | some | no | no | no | some | no | no | no | no |
| Mannitol | some | some | no | no | no | no | some | no | no | no |

Replicate 3

| Carrier | RT | 75° C. | 100° C. | 120° C. | 135° C. |
|---|---|---|---|---|---|
| Oat Com | yes | yes | some | no | no |
| Oat Silk | yes | yes | yes | yes | some |
| DermiVeil | no | no | no | no | no |
| Starch | some | some | no | no | no |
| Mannitol | some | no | no | no | no |

### Luciferase Glow Assay

24.8 mg of firefly luciferase coated powder was resuspended in Tris buffer and incubated for 2 h in an overhead rotator at 10 rpm and 4° C. for reconstitution of the protein. The solid particles were pelleted and the supernatant was used for a glow assay. Oxidation of d-Luciferin by the enzyme can be measured as luminescence. Luciferase coated on Oat Com™ and Oat Silk™ remained active even after exposure to 135° C. for 1 h (Table 10). Activity of the protein coated onto DermiVeil™, starch or mannitol was reduced or lost between 100° C. and 120° C.

TABLE 10
Summary table indicating activity of the firefly luciferase coated on different carriers and
exposed to temperatures between 75° C. and 135° C. for 1 h. The activity is coded:
yes = high luminescence signal; some = medium luminescence signal; no = no luminescence signal.

| | Replicate 1 | | | | | Replicate 2 | | | | |
|---|---|---|---|---|---|---|---|---|---|---|
| Carrier | RT | 75° C. | 100° C. | 120° C. | 135° C. | RT | 75° C. | 100° C. | 120° C. | 135° C. |
| Oat Com | yes | yes | yes | yes | yes | yes | yes | yes | yes | yes |
| Oat Silk | yes | yes | yes | yes | yes | yes | yes | yes | yes | yes |
| DermiVeil | some | yes | yes | some | no | some | yes | some | no | no |
| Starch | yes | yes | yes | yes | some | some | yes | yes | some | no |
| Mannitol | yes | yes | yes | some | no | yes | yes | some | some | no |

Replicate 3

| Carrier | RT | 75° C. | 100° C. | 120° C. | 135° C. |
|---|---|---|---|---|---|
| Oat Com | yes | yes | yes | yes | yes |
| Oat Silk | yes | yes | yes | yes | yes |
| DermiVeil | some | some | no | some | no |
| Starch | some | some | yes | some | no |
| Mannitol | no | some | no | no | no |

### β-Galactosidase

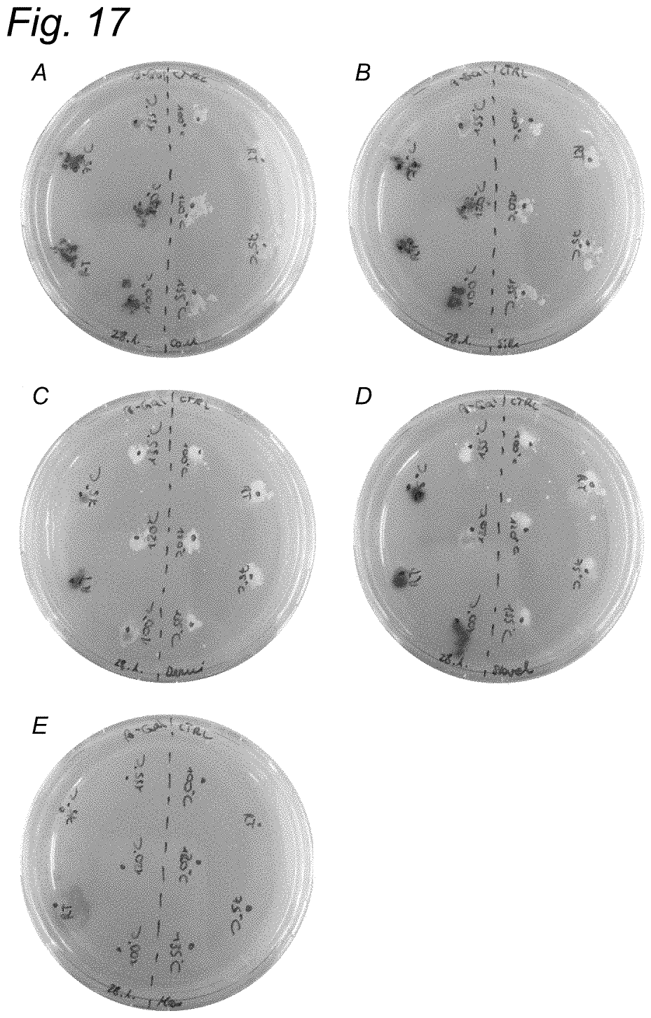

Remaining activity of β-galactosidase coated onto different carriers and exposed to different temperatures was tested by directly spotting it on chromogenic coliform agar. Hydrolysis of the compound Salmon-β-d-galactosidase present in the media catalyzed by the β-galactosidase leads to red stain on the site of activity. β-galactosidase coated on Oat Com™ and Oat Silk™ showed full activity after one hour at 120° C. and some residual activity at 135° C. (FIG. 17A, 17B). When starch was used as carrier, full activity was retained at room temperature, 75° C. and 100° C. At 120° C. β-galactosidase activity was decreased and at 135° C. completely lost (FIG. 17D). β-galactosidase on DermiVeil™ showed activity at room temperature and some residual activity at 75° C. and 100° C. (FIG. 17C). Mannitol did not confer any heat stability when used as a carrier for β-galactosidase (FIG. 17E). Therefore, activity was only observed for samples at room temperature.

Activity of β-galactosidase coated onto different carriers and exposed to high temperatures is summarized for each biological replicate in Table 11.

TABLE 11
Summary table indicating activity of the β-galactosidase coated on different
carriers and exposed to temperatures between 75° C. and 135° C.
for 1 h. The activity is coded: yes = red stain at site of powder spotting;
some = some faint color formation at site of powder spotting; no = no color formation.

| | Replicate 1 | | | | | Replicate 2 | | | | |
|---|---|---|---|---|---|---|---|---|---|---|
| Carrier | RT | 75° C. | 100° C. | 120° C. | 135° C. | RT | 75° C. | 100° C. | 120° C. | 135° C. |
| Oat Com | yes | yes | yes | yes | some | yes | yes | yes | yes | some |
| Oat Silk | yes | yes | yes | yes | some | yes | yes | yes | yes | some |
| DermiVeil | yes | yes | some | no | no | yes | yes | some | no | no |
| Starch | yes | yes | yes | some | no | yes | yes | yes | some | no |
| Mannitol | yes | no | no | no | no | yes | no | no | no | no |

Replicate 3

| Carrier | RT | 75° C. | 100° C. | 120° C. | 135° C. |
|---|---|---|---|---|---|
| Oat Com | yes | yes | yes | yes | some |
| Oat Silk | yes | yes | yes | yes | some |
| DermiVeil | yes | yes | some | no | no |
| Starch | yes | yes | yes | very | no |
| | | | | little | |
| Mannitol | no | no | no | no | no |

### DISCUSSION AND CONCLUSION

In this study different enzymes were coated on carriers via a lyophilisation process. Especially the two oatmeal derived powders Oat Com™ and Oat Silk™ showed improved activity preservation of the proteins even when exposed to high temperatures. Even though residual powder particles led to fluctuations in the OD_{600 nm} measurements of the turbidity reduction assays, clear lytic activity was observed up to 130° C. and 135° C. for XZ.700 and HPL511, respectively. Starch appeared to be a good carrier for certain proteins. Due to very poor activity on preservation and its hygroscopic tendency, sucrose was only tested with XZ.700 and excluded from further experiments. In general, the firefly luciferase seemed less susceptible to the heat treatment compared to the other proteins tested. In contrast, the lyophilisation procedure seemed to harm the horseradish peroxidase the most. Overall, this technique worked surprisingly well to preserve enzymatic activity of a wide range of proteins. The tendency of proteins to lose activity in an aqueous solution could be surpassed by storing them in a solid form until usage (Manning, Patel et al. 1989). This technique may work especially well on skin as the site of treatment would provide the moisture necessary for reconstitution of the protein. Additionally, using oatmeal as a carrier would be beneficial for many skin conditions due to its anti-inflammatory and anti-itchy properties (Fowler 2014).

### REFERENCES

Nedorost, Susan T. (2012). Generalized Dermatitis in Clinical Practice. Springer Science & Business Media. pp. 1-3, 9, 13-14.
Handout on Health: Atopic Dermatitis (A type of eczema)”. NIAMS. May 2013.
McAleer, M A; Flohr, C; Irvine, A D (23 Jul. 2012). “Management of difficult and severe eczema in childhood” BMJ (Clinical Research Ed.).
GBD 2015 Disease and Injury Incidence and Prevalence, Collaborators. (8 Oct. 2016). “Global, regional, and national incidence, prevalence, and years lived with disability for 310 diseases and injuries, 1990-2015: a systematic analysis for the Global Burden of Disease Study 2015”. Lancet. 388 (10053): 1545-1602.
Habif (2015). Clinical Dermatology (6 ed.). Elsevier Health Sciences. p. 171.
Mowad, C M; Anderson, B; Scheinman, P; Pootongkam, S; Nedorost, S; Brod, B (June 2016). “Allergic contact dermatitis: Patient management and education”. Journal of the American Academy of Dermatology. 74 (6): 1043-54.
Lurati, A R (February 2015). “Occupational risk assessment and irritant contact dermatitis”. Workplace Health & Safety. 63 (2): 81-88.
Denyes, J. M., M. Dunne, S. Steiner, M. Mittelviefhaus, A. Weiss, H. Schmidt, J. Klumpp and M. J. Loessner (2017). “Modified Bacteriophage S16 Long Tail Fiber Proteins for Rapid and Specific Immobilization and Detection of Salmonella Cells.” Appl Environ Microbiol 83(12).
Eugster, M. R. and M. J. Loessner (2012). “Wall teichoic acids restrict access of bacteriophage endolysin Ply118, Ply511, and PlyP40 cell wall binding domains to the Listeria monocytogenes peptidoglycan.” J Bacteriol 194(23): 6498-6506.
Fowler, J. F., Jr. (2014). “Colloidal oatmeal formulations and the treatment of atopic dermatitis.” J Drugs Dermatol 13(10): 1180-1183; quiz 1184-1185.
Manning, M. C., K. Patel and R. T. Borchardt (1989). “Stability of protein pharmaceuticals.” Pharm Res 6(11): 903-918.
Schmelcher, M., D. M. Donovan and M. J. Loessner (2012). “Bacteriophage endolysins as novel antimicrobials.” Future Microbiol 7(10): 1147-1171.

1. A method for stabilizing a protein of interest, comprising contacting the protein with a cereal meal or variant thereof, wherein the protein of interest is an enzyme.

2. A non-aqueous composition comprising a protein of interest and a cereal meal or variant thereof, wherein the protein of interest is an endolysin.

3. (canceled)

4. (canceled)

5. The non-aqueous composition according to claim 2, wherein the endolysin is specific for Staphylococcus, preferably Staphylococcus aureus.

6. The non-aqueous composition according to claim 2, wherein the cereal meal or variant thereof comprises in weight between about 50% to about 85% carbohydrates, between about 10 and about 25% protein, between about 0% and about 12% lipids, between about 0% and about 10% beta-glucans and between about 0% and about 15% fibre.

7. The non-aqueous composition according to claim 2, wherein the cereal is selected from the group consisting of maize, rice, wheat, barley, sorghum, millet, oats, rye, triticale, quinoa, spelt and fonio.

8. The non-aqueous composition according to claim 2, wherein the cereal meal is oat meal, preferably colloidal oat meal, more preferably Oat Com™, Oat Silk™, or DermiVeil™.

9. The non-aqueous composition according to claim 2, wherein the protein of interest and the cereal meal or variant thereof are mixed in an aqueous liquid, which is subsequently lyophilized.

10. (canceled)

11. (canceled)

12. The non-aqueous composition according to claim 2, wherein the composition is a cream.

13. A method of treatment of atopic dermatitis comprising administration of the non-aqueous composition according to claim 2 to a subject in need thereof.

14. (canceled)

15. (canceled)

16. The method according to claim 1, wherein the endolysin is specific for Staphylococcus, preferably Staphylococcus aureus.

17. The method according to claim 1, wherein the cereal meal or variant thereof comprises in weight between about 50% to about 85% carbohydrates, between about 10 and about 25% protein, between about 0% and about 12% lipids, between about 0% and about 10% beta-glucans and between about 0% and about 15% fibre.

18. The method according to claim 1, wherein the cereal is selected from the group consisting of maize, rice, wheat, barley, sorghum, millet, oats, rye, triticale, quinoa, spelt and fonio.

19. The method according to claim 1, wherein the cereal meal is oat meal, preferably colloidal oat meal, more preferably Oat Com™, Oat Silk™, or DermiVeil™.

20. The method according to claim 1, wherein the protein of interest and the cereal meal or variant thereof are mixed in an aqueous liquid, which is subsequently lyophilized.


SEQUENCE LISTING (75 sequences)

SEQ ID NO: 1 - DNA, 1461 bp, Staphylococcus bacteriophage 2638A
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 2 - protein, 486 aa, Staphylococcus bacteriophage 2638A
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 3 - DNA, 279 bp, Staphylococcus simulans
tggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 4 - protein, 92 aa, Staphylococcus simulans
WKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 5 - DNA, 285 bp, Staphylococcus bacteriophage 2638A
tggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 6 - protein, 94 aa, Staphylococcus bacteriophage 2638A
WKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 7 - DNA, 282 bp, Staphylococcus bacteriophage phiNM3
ggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttt

SEQ ID NO: 8 - protein, 94 aa, Staphylococcus bacteriophage phiNM3
GKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 9 - DNA, 585 bp, Staphylococcus bacteriophage K
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaat

SEQ ID NO: 10 - protein, 195 aa, Staphylococcus bacteriophage K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKN

SEQ ID NO: 11 - DNA, 420 bp, Staphylococcus bacteriophage Twort
atgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttatt

SEQ ID NO: 12 - protein, 140 aa, Staphylococcus bacteriophage Twort
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFI

SEQ ID NO: 13 - DNA, 438 bp, Staphylococcus bacteriophage 2638A
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagat

SEQ ID NO: 14 - protein, 146 aa, Staphylococcus bacteriophage 2638A
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKD

SEQ ID NO: 15 - DNA, 420 bp, Staphylococcus simulans
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatat

SEQ ID NO: 16 - protein, 140 aa, Staphylococcus simulans
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGY

SEQ ID NO: 17 - DNA, 510 bp, Staphylococcus bacteriophage 2638A
ggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggt

SEQ ID NO: 18 - protein, 169 aa, Staphylococcus bacteriophage 2638A
NKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDG

SEQ ID NO: 19 - DNA, 1407 bp, Artificial Sequence
  artificial construct
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 20 - protein, 468 aa, Artificial Sequence
  artificial construct
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 21 - DNA, 1209 bp, Artificial Sequence
  artificial construct
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 22 - protein, 402 aa, Artificial Sequence
  artificial construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 23 - DNA, 1686 bp, Artificial Sequence
  artificial construct
gcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 24 - protein, 561 aa, Artificial Sequence
  artificial construct
AKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 25 - DNA, 1131 bp, Artificial Sequence
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 26 - protein, 376 aa, Artificial Sequence
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 27 - DNA, 1125 bp, Artificial Sequence
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 28 - protein, 374 aa, Artificial Sequence
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 29 - DNA, 1131 bp, Artificial Sequence
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 30 - protein, 376 aa, Artificial Sequence
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 31 - DNA, 975 bp, Artificial Sequence
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 32 - protein, 324 aa, Artificial Sequence
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 33 - DNA, 912 bp, Artificial Sequence
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 34 - protein, 303 aa, Artificial Sequence
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 35 - DNA, 966 bp, Artificial Sequence
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 36 - protein, 321 aa, Artificial Sequence
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 37 - DNA, 927 bp, Artificial Sequence
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 38 - protein, 308 aa, Artificial Sequence
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 39 - DNA, 921 bp, Artificial Sequence
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 40 - protein, 306 aa, Artificial Sequence
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 41 - DNA, 927 bp, Artificial Sequence
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 42 - protein, 308 aa, Artificial Sequence
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 43 - DNA, 843 bp, Artificial Sequence
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 44 - protein, 280 aa, Artificial Sequence
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 45 - DNA, 837 bp, Artificial Sequence
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 46 - protein, 278 aa, Artificial Sequence
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 47 - DNA, 843 bp, Artificial Sequence
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 48 - protein, 280 aa, Artificial Sequence
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 49 - DNA, 1755 bp, Artificial Sequence
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 50 - protein, 584 aa, Artificial Sequence
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 51 - DNA, 1749 bp, Artificial Sequence
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 52 - protein, 582 aa, Artificial Sequence
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 53 - DNA, 1755 bp, Artificial Sequence
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 54 - protein, 584 aa, Artificial Sequence
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 55 - DNA, 1533 bp, Artificial Sequence
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 56 - protein, 510 aa, Artificial Sequence
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 57 - DNA, 1470 bp, Artificial Sequence
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 58 - protein, 489 aa, Artificial Sequence
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 59 - DNA, 1524 bp, Artificial Sequence
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 60 - protein, 507 aa, Artificial Sequence
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 61 - DNA, 1446 bp, Artificial Sequence
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 62 - protein, 481 aa, Artificial Sequence
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 63 - DNA, 1440 bp, Artificial Sequence
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 64 - protein, 479 aa, Artificial Sequence
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 65 - DNA, 1446 bp, Artificial Sequence
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 66 - protein, 481 aa, Artificial Sequence
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 67 - DNA, 1278 bp, Artificial Sequence
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 68 - protein, 425 aa, Artificial Sequence
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 69 - DNA, 1272 bp, Artificial Sequence
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 70 - protein, 423 aa, Artificial Sequence
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 71 - DNA, 1278 bp, Artificial Sequence
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 72 - protein, 425 aa, Artificial Sequence
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 73 - protein, 506 aa, Artificial Sequence
  artificial construct
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 74 - protein, 462 aa, Artificial Sequence
  artificial construct
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGLKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 75 - protein, 10 aa, Artificial Sequence
  Synthetic peptide
ELAGIGILTV